(19) United States
(12) Patent Application Publication
(10) Pub. No.: US 2026/0036595 A1
(43) Pub. Date: Feb. 5, 2026

(54) STRUCTURE-BASED PEPTIDES FOR DETECTION OF TRANSTHYRETIN AMYLOID FIBRILS AND AGGREGATES IN PREECLAMPSIA

(71) Applicant: THE BOARD OF REGENTS OF THE UNIVERSITY OF TEXAS SYSTEM, Austin, TX (US)

(72) Inventors: Lorena Saelices Gomez, Flower Mound, TX (US); Rose Pedretti, Dallas, TX (US)

(73) Assignee: THE BOARD OF REGENTS OF THE UNIVERSITY OF TEXAS SYSTEM, Austin, TX (US)

(21) Appl. No.: 19/361,684

(22) Filed: Oct. 17, 2025

Related U.S. Application Data
(63) Continuation-in-part of application No. 18/875,065.
(60) Provisional application No. 63/352,521, filed on Jun. 15, 2022.
(60) Provisional application No. 63/382,122, filed on Nov. 3, 2022.
(60) Provisional application No. 63/708,957, filed on Oct. 18, 2024.

(51) Int. Cl. G01N 33/68

(52) U.S. Cl. CPC G01N 33/689; G01N 2800/368; G01N 2800/50; G01N 2800/52

(57) ABSTRACT

Provided are methods of evaluating risk for, diagnosing, and/or treating preeclampsia in a subject based on levels of transthyretin fibrils, oligomers or aggregates that can be detected using structure-specific polypeptide probes selective for aggregated transthyretin. Also provided are methods of monitoring the efficacy of a therapeutic administered to treat preeclampsia using these polypeptide probes.

### CROSS-REFERENCE TO RELATED APPLICATIONS

This application claims the benefit of U.S. Provisional Application No. 63/708,957, filed on Oct. 18, 2024, and is a continuation in part of U.S. application Ser. No. 18/875,065, which is a national stage application filed under 35 U.S.C. § 371 of International Patent Application No. PCT/US2023/068432 entitled “STRUCTURE-BASED PROBE FOR DETECTION OF TRANSTHYRETIN AMYLOID FIBRILS AND AGGREGATES” filed Jun. 14, 2023, which claims the benefit of U.S. Provisional Application No. 63/382,122, filed on Nov. 3, 2022 and U.S. Provisional Application No. 63/352,521 filed on Jun. 15, 2022. The disclosures of U.S. Provisional Application No. 63/708,957, U.S. application Ser. No. 18/875,065, International Patent Application No. PCT/US2023/068432, U.S. Provisional Application No. 63/352,521, and U.S. Provisional Application No. 63/382,122 are each are hereby incorporated by reference in their entireties.

### ACKNOWLEDGEMENT OF GOVERNMENT SUPPORT

This invention was made with government support under Grant No. HL163810 awarded by the National Institutes of Health. The government has certain rights in the invention.

### SEQUENCE LISTING

The instant application contains a Sequence Listing which has been submitted electronically in XML format and is hereby incorporated by reference in its entirety for all purposes. The XML copy, created on Oct. 13, 2025, is titled UTSD.P4057US.CP1_SequenceListing.xml and is 41,476 bytes in size.

### BACKGROUND

### 1. Field

The present inventive concept is directed to methods of diagnosing and treating preeclampsia based on levels of transthyretin amyloid fibrils and aggregates.

### 2. Discussion of Related Art

Amyloid diseases are characterized by aggregation of particular proteins into amyloid fibrils, which then may serve as seeds to induce further aggregation of a parental protein. One major protein that may be prone to aggregation is transthyretin (TTR). Transthyretin (TTR) is a 55 kDa tetrameric protein that transports retinol binding protein (RBP) and thyroxine (T4) in the blood and cerebrospinal fluid. Amyloid aggregation of TTR occurs by dissociation of tetrameric TTR into monomers; these partially unfold into amyloidogenic intermediates, and self-associate into soluble oligomers and amyloid aggregates. Familial point mutations are known to destabilize the tetramer, leading to a faster dissociation and consequent amyloid aggregation.

Preeclampsia (PE) is a pregnancy-specific vascular syndrome that emerges after 20 weeks of gestation and is a major cause of maternal, fetal and neonatal morbidity and mortality. Despite affecting approximately 2-8% of pregnancies worldwide, PE still lacks reliable, early diagnosis or therapy. Transthyretin (TTR) aggregates have been detected in the placenta of preeclamptic patients. These aggregates could represent a causal pathway for preeclampsia.

The present disclosure is based, in part, on the surprising discovery that levels of TTR aggregates and fibrils are elevated in preeclampsia patients. This discovery, together with disclosed methods of detecting aggregates and fibrils, may lead to improved diagnosis and treatment of this patient population.

### SUMMARY

The present disclosure is based, at least in part, on the discovery of aggregated transthyretin as a potential biomarker or indicator of preeclampsia.

Accordingly, aspects of the present disclosure provide methods of determining whether a subject is at risk of preeclampsia, the methods comprising: (a) detecting a transthyretin oligomer, fibril or molecule in a sample obtained from the subject, and (b) identifying the subject as at risk for preeclampsia if the transthyretin oligomer, fibril or molecule is detected in the sample. In some aspects, the subject is determined to be at risk of preeclampsia if a level of transthyretin oligomer, fibril or molecule detected in the sample exceeds a threshold.

Further aspects of the present disclosure provide methods of diagnosing a subject with preeclampsia, the method comprising: (a) detecting a transthyretin oligomer, fibril or molecule in a sample obtained from the subject and (b) diagnosing the subject with preeclampsia if the transthyretin oligomer, fibril or molecule is detected in the sample. In some aspects, the subject is diagnosed with preeclampsia or if a level of transthyretin oligomer, fibril or molecule detected in the sample exceeds a threshold.

Further aspects of the present disclosure provide for methods of monitoring an effectiveness of a therapeutic administered to a subject to treat a preeclampsia, the method comprising (a) detecting an oligomer, aggregate or fibril of transthyretin in a first sample obtained from the subject, b) administering the therapeutic to the subject, and (c) detecting an oligomer, aggregate or fibril of transthyretin in a second sample obtained from the sample after the therapeutic is administered, wherein the therapeutic is determined to be effective if fewer oligomers, aggregates and/or fibrils of transthyretin are detected in the second sample compared to the first sample. In some aspects, the method further comprises monitoring more than one dose of the therapeutic to identify an effective amount of the therapeutic, wherein the effective amount of the therapeutic results in a largest reduction in the detection of oligomers, aggregates, and/or fibrils of transthyretin in the second sample compared to the first sample. In any of these methods, the subject may have been determined to be at risk for preeclampsia and/or diagnosed with preeclampsia according to any method disclosed herein.

Further aspects of the present disclosure provide for methods of method of treating a subject for preeclampsia, the method comprising administering an effective amount of a therapeutic to the subject, wherein (a) the subject has been determined to be at risk for preeclampsia, (b) the subject has been diagnosed with preeclampsia, and/or (c) the effective amount of the therapeutic is determined according any of the methods disclosed herein.

In various aspects, a therapeutic administered to a subject according to any of the disclosed methods comprises an inhibitor of transthyretin expression and/or aggregation. In some aspects, the therapeutic comprises a small molecule, a gene silencer or an antibody. In some aspects, the therapeutic comprises tafamidis.

In any of the disclosed methods herein, the step of detecting a transthyretin oligomer, fibril or molecule in a sample can comprise (a) contacting a sample first with a polypeptide probe selective for aggregated transthyretin, (b) allowing the polypeptide probe to bind any oligomers, aggregates or fibrils of transthyretin in the sample, and (c) detecting a complex comprising the polypeptide probe and an oligomer, aggregate or fibril of transthyretin, wherein the presence of the complex correlates to the presence of an oligomer, aggregate or fibril of transthyretin in the sample.

In various aspects, the polypeptide probe selective for aggregated transthyretin comprises first peptide comprising the sequence HVAHPFVEFTE (SEQ ID NO: 1) and a second peptide comprising the sequence SYVTNPTSYAVT (SEQ ID NO: 2), wherein the first and second peptide are covalently linked via a linker peptide and wherein the polypeptide probe further comprises a detectable label.

In various aspects, the linker peptide comprises the sequence GGGSTE (SEQ ID NO: 3), EAAAK (SEQ ID NO: 4), PAPAP (SEQ ID NO: 5) or GGGGGG (SEQ ID NO: 6).

In further aspects, the polypeptide probe my further comprise an epitope tag that facilitates solubility, manipulation and/or purification of the polypeptide. In some aspects, the epitope tag comprises a peptide that increases the affinity of the polypeptide to an affinity column. In further aspects, the epitope tag comprises a plurality of histidine or lysine residues. For example, in various aspects, the epitope tag comprises a peptide consisting of the sequence DYKDDDDK (SEQ ID NO: 7) or YPYDVPDYA (SEQ ID NO: 8). In some instances, the epitope tag increases the solubility of the polypeptide. For example, in some aspects, the epitope tag comprises a plurality of arginine residues.

In various aspects, the detectable label is covalently linked to the N-terminus of the first peptide or to the C-terminus of the second peptide. In various aspects, the detectable label is covalently linked to polypeptide probe via a linker (e.g., aminohexanoic acid (Ahx)).

In any of these aspects, the detectable label can comprise tetramethylrhodamine (TAMRA), Fluorescein isothiocyanate (FITC), aminohexanoic acid (Ahx) or any combination thereof.

In any of these aspects, the polypeptide probe can comprise the amino acid sequence RRRRHVAHPFVEFTEGGGSTERRRRSYVTNPTSYAVT (SEQ ID NO: 9). In some aspects, the polypeptide probe comprises the amino acid sequence YPYDVPDYARRRRHVAHPFVEFTEGGGSTERRRRSYVTNPTSYAVT (SEQ ID NO: 10), DYKDDDDK-RRRRHVAHPFVEFTEGGGSTERRRRSYVTNPTSYAVT (SEQ ID NO: 11), or HHHHHHRRRRHVAHPFVEFTEGGGSTERRRRSYVTNPTSYAVT (SEQ ID NO: 12). In still further aspects, the polypeptide probe is selected from the group consisting of: TAMRA-YPYDVPDYA-RRRRHVAHPFVEFTEGGGSTERRRRSYVTNPTSYAVT (SEQ ID NO: 14), TAMRA-DYKDDDDK-RRRRHVAHPFVEFTEGGGSTERRRRSYVTNPTSYAVT (SEQ ID NO: 15), TAMRA-HHHHHH-RRRRHVAHPFVEFTEGGGSTERRRRSYVTNPTSYAVT (SEQ ID NO: 16), FITC-Ahx-YPYDVPDYA-RRRRHVAH PFVEFTEGGGSTERRRRSYVTNPTSYAVT (SEQ ID NO: 17), FITC-Ahx-DYKDDDDK-RRRRHVAHPFVEFTEGGGSTERRRRSYVTNPTSYAVT (SEQ ID NO: 18), FITC-Ahx-HHHHHH-RRRRHVAHPFVEFTEGGGSTERRRRSYVTNPTSYAVT (SEQ ID NO: 19), FITC-Ahx-RRRRHVAHPFVEFTEGGGSTERRRRSYVTNPTSYAVT (SEQ ID NO: 20) wherein TAMRA is tetramethylrhodamine, FITC is Fluorescein isothiocyanate (FITC) and Ahx is an aminohexanoic acid linker. For example, in some aspects, the polypeptide probe is FITC-Ahx-HHHHHH-RRRRHVAHPFVEFTEGGGSTERRRRSYVTNPTSYAVT (SEQ ID NO: 19), FITC-Ahx-YPYDVPDYA-RRRRHVAHPFVEFTEGGGSTERRRRSYVTNPTSYAVT (SEQ ID NO: 17), or FITC-Ahx-DYKDDDDK-RRRRHVAHPFVEFTEGGGSTERRRRSYVTNPTSYAVT (SEQ ID NO: 18), wherein FITC is Fluorescein isothiocyanate (FITC) and Ahx is an aminohexanoic acid linker. As an example, the polypeptide probe may be FITC-Ahx-H HHHHH-RRRRHVAH PFVEFTEGGGSTERRRRSYVTNPTSYAVT (SEQ ID NO: 19), wherein FITC is Fluorescein isothiocyanate (FITC) and Ahx is an aminohexanoic acid linker.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein. Embodiments of the present disclosure are illustrated by way of example in which like reference numerals indicate similar elements and in which:

FIG. 1 is an illustrative schematic of a transthyretin (TTR) fibril and a peptide probe of the present disclosure.

FIG. 2 is an illustrative schematic of an immunoblot procedure to test the efficacy of the disclosed peptide probes to detect TTR fibrils or aggregates in a sample.

FIG. 3 is an illustrative dot blot showing detection of ATTR fibrils from different samples using the peptide probe of the present disclosure (0.5 μg sample and 1 nM peptide).

FIG. 4 is an illustrative dot blot showing detection of ATTR fibrils using 1 fM of the disclosed peptide probe and 10-500 ng of a sample.

FIG. 5 is an illustrative dot blot showing detection of ATTR fibrils from ATTR heart lysates, using 1 nM of the peptide probe (0.5 μg sample and 1 nM peptide).

FIG. 6 is an illustrative dot blot showing detection of ATTR fibrils in ATTR serum.

FIG. 7 is a schematic illustrating a protocol to measure peptide binding to target fibrils using nanogold transmission electron microscopy.

FIGS. 8A and 8B depict illustrative scanning electron micrographs of ATTRwt fibrils labeled with 5 μM peptide probes using 50 nM Ni-NTA Nanogold (FIG. 8A) or 250 nM Ni-NTA Nanogold (FIG. 8B)

FIGS. 9A and 9B depict illustrative scanning electron micrographs of Tau fibrils incubated with 5 μM peptide probes and 50 nM Ni-NTA Nanogold. FIG. 9B is a larger image of the inset in FIG. 9A.

FIG. 10 illustrates potential mechanisms of binding for the polypeptides of the present disclosure.

FIG. 11 depicts illustrative dot blots of samples probed with a polypeptide probe of the present disclosure before or after denaturing conditions.

FIG. 12 depicts plots of relative fluorescence correlating to binding of a polypeptide probe to ATTR fibrils in patient serum and plasma samples before and after treatment.

FIGS. 13A and 13B depict an illustrative immunoblot (FIG. 13A) and quantitation (FIG. 13B) of ex-vivo ATTR cardiac fibrils, heart lysates, recombinant protein, and control samples probed with a polypeptide probe.

FIGS. 14A and 14B depict illustrative immunoblots of ATTR fibrils and lysates probed with either an anti-TTR antibody (FIG. 14A) or a polypeptide probe (FIG. 14B) under nondenaturing conditions.

FIG. 15 depicts an illustrative dot blot of ATTR fibrils and lysates before and after denaturation probed with a polypeptide probe.

FIGS. 16A-16C depict a schematic (FIG. 16A) of a rational design workflow for development of polypeptide probes, thioflavin T (ThT) screening of third generation transthyretin amyloid binders (Tabs) to assess candidate peptide binding to fibrils (FIG. 16B) and assessment of polypeptide probe fibril binding activity through ThT assay (FIG. 16C).

FIG. 17 illustrates an experimental workflow for dot blotting to quantify polypeptide probe binding to samples of interest. Recombinant protein or patient samples (extracted fibrils from heart, cardiac lysates, or blood) are applied to nitrocellulose membrane then incubated with TAD peptide. TAD binding to sample is measured through relative fluorescence intensity, which can then be quantified to provide insight into the relative fibrillar content of each sample.

FIGS. 18A and 18B depict an illustrative dot blot and quantification of TAD1 fluorescence intensity of binding to extracted ATTR fibrils and controls (FIG. 18A) and ATTR fibrils in cardiac lysates and controls (FIG. 18B).

FIGS. 19A-19D depict titrated ATTRwt fibrils probed with TAD1 (FIG. 19A), quantification of TAD1 fluorescence intensity corresponding to amount of fibrils in FIG. 19A (FIG. 19B), a precision assay to determine whether TAD1 can detect small variations in ATTR fibrils loaded onto membrane (FIG. 19C) and quantification of TAD1 fluorescence intensity of binding to each dot in FIG. 19C (FIG. 19D).

FIGS. 20A-20C show that TAD1 detects unique ATTR species in plasma but not serum and depict dot blotting of recombinant protein controls and extracted ATTRwt fibrils (top row), serum (middle row) and plasma (bottom row) from ATTRv amyloidosis patients before and after treatment with gene silencers, and a healthy age-matched individual (FIG. 20A) and quantification of TAD1 fluorescence intensity of binding to serum samples (FIG. 20B) and plasma samples (FIG. 20C).

FIGS. 21A-21B show that TAD1 detects unique ATTR species in plasma of cardiac ATTR amyloidosis patients and depicts TAD1 fluorescence intensity of negative controls (including one light chain amyloidosis (AL amyloidosis) patient and 32 healthy age-matched individuals), ATTRv carriers with no cardiac phenotype, ATTR amyloidosis patients before starting treatment, and ATTR amyloidosis patients on treatment (FIG. 21A) and TAD1 fluorescence intensity comparing negative controls, ATTR amyloidosis patients, and a larger cohort of AL amyloidosis patients (FIG. 21B).

FIGS. 22A-22D show that TAD1 binds large species from recombinant protein, ATTR fibril extracts, and plasma and depicts dot blotting of recombinant transthyretin aggregation over time probing for total protein (top), aggregated species in total sample with TAD1 (middle), and insoluble components after centrifugation (bottom) (FIG. 22A), native gel electrophoresis of TAD1, recombinant TTR and amyloid extracted from ATTR amyloidosis patients, blotted and probed with an anti-TTR antibody (top) and TAD1 (bottom) (FIG. 22B), a scheme for a filtration experiment (FIG. 22C) and a dotblot of Filtered plasma of ATTR amyloidosis patients and healthy age matched controls probing with an anti-TTR antibody (left) and TAD1 (right) (FIG. 22D).

FIGS. 23A-23B show that TAD1 binds ATTR fibrils in a conformation-dependent manner and depicts a Native gel electrophoresis of TAD1, recombinant TTR and amyloid extracted from ATTR amyloidosis patients, blotted and probed with an anti-TTR antibody (left) and TAD1 (right) (FIG. 23A) and a gel electrophoresis of same samples under denaturing conditions, blotted and probed with an anti-TTR antibody (left) and TAD1 (right) (FIG. 23B).

FIGS. 24A-24D show a gel shift assay of ATTRwt patient plasma and healthy plasma with increasing concentrations of TAD1, probed with an anti-TTR antibody (FIG. 24A) and quantification of transthyretin signal from gel shift assay in wells (FIG. 24B), oligomers (FIG. 24C), and tetramers (FIG. 24D).

FIGS. 25A-25B show that TAD1 binds ATTR via a unique mechanism and depicts quantification of TAD1 binding to ATTRwt fibrils in different pH buffers (FIG. 25A) and different salt concentrations (FIG. 25B).

FIGS. 26A-26B show a scheme for labeling experiments (FIG. 26A) and nanogold labeling of ATTRwt fibrils (FIG. 26B) or tau fibrils extracted from brain (FIG. 26C) using TAD1 and nanogold (50 and 250 μM for ATTRwt fibrils and 50 μM for tau).

FIGS. 27A-27B show an illustrative dot blot (FIG. 27A) and quantification (FIG. 27B) of transthyretin species probed using TAD1 in placental lysates.

FIGS. 28A-28C show an illustrative dot blot (FIG. 28A) labeled with TAD1, a dot blot labeled with anti-TTR (FIG. 28B) and quantification (FIG. 28C) of TAD1 vs anti-TTR signal in different patient samples.

FIG. 29 shows a schematic of a fibril extraction workflow according to some aspects of the present disclosure.

FIG. 30 shows an illustrative dot blot labeled with TAD1 of different patient samples.

FIG. 31 shows illustrative images of TTR fibrils extracted from preeclampsia patient samples.

FIGS. 32A-32B show an illustrative dot blot (FIG. 32A) and quantification (FIG. 32B) of TTR fibrils labeled with TAD1 from patient samples.

### DETAILED DESCRIPTION

The following detailed description references the accompanying drawings that illustrate various embodiments of the present disclosure. The drawings and description are intended to describe aspects and embodiments of the present disclosure in sufficient detail to enable those skilled in the art to practice the present disclosure. Other components can be utilized and changes can be made without departing from the scope of the present disclosure. The following description is, therefore, not to be taken in a limiting sense. The scope of the present disclosure is defined only by the appended claims, along with the full scope of equivalents to which such claims are entitled.

The present disclosure is based, at least in part, on the surprising discovery that aggregated transthyretin is elevated in preeclampsia patients. The instant disclosure, therefore, is based on the use of aggregated transthyretin as a biomarker for preeclampsia and the use of detection probes selective for transthyretin aggregates as diagnostic tools for identifying and treating patients with this disorder.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present disclosure or the appended claims.

Further, as the present disclosure is susceptible to embodiments of many different forms, it is intended that the present disclosure be considered as an example of the principles of the present disclosure and not intended to limit the present disclosure to the specific embodiments shown and described. Any one of the features of the present disclosure may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the present disclosure may include a variety of combinations and/or integrations of the embodiments described herein. Additionally, all aspects of the present disclosure, as described herein, are not essential for its practice. Likewise, other systems, methods, features, and advantages of the present disclosure will be, or become, apparent to one with skill in the art upon examination of the figures and the description. It is intended that all such additional systems, methods, features, and advantages be included within this description, be within the scope of the present disclosure, and be encompassed by the claims.

As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

The terms “comprising,” “including,” “encompassing” and “having” are used interchangeably in this disclosure. The terms “comprising,” “including,” “encompassing” and “having” mean to include, but not necessarily be limited to the things so described.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

As used herein, the terms “treat”, “treating”, “treatment” and the like, unless otherwise indicated, can refer to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the condition, or disorder.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mo/. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

It should also be understood that, unless clearly indicated to the contrary, in any methods claimed herein that include more than one step or act, the order of the steps or acts of the method is not necessarily limited to the order in which the steps or acts of the method are recited.

### II. Compositions

The present disclosure provides for compositions for detecting transthyretin (TTR) oligomers or aggregates. In various aspects, the compositions comprise at least one polypeptide probe comprising a first peptide and a second peptide, wherein the first and second peptide have affinity for a TTR oligomer or aggregate and are covalently linked via a linker peptide, wherein the polypeptide is covalently linked to a detectable label at an N or C terminus.

### 1. Components of Polypeptide Probes

### a) Transthyretin Binding Peptides

Transthyretin fibrils are known to have multiple structural domains, identified in the art as strands A to G. In various aspects, the first and/or second peptide of the polypeptide probe may bind to any of these strands. In various aspects, the first and second peptide of the polypeptide probe may bind to different strands (e.g., to strand “F” or strand “H”). In various aspects, the first and second peptide of the polypeptide probe may bind to different (distinct) strand of the TTR oligomer/aggregate simultaneously. In some aspects, the first peptide of the polypeptide probe may bind a region of the TTR fibril (e.g., a strand or a loop) comprising a peptide selected from VAVHVF (SEQ ID NO: 22, Strand B), IYKVEI (SEQ ID NO: 23, Strand E), KALGIS (SEQ ID NO: 24, loop connecting Strands E and F), AEVVFT, SEQ ID NO: 25; Strand F), YTIAAL, SEQ ID NO: 26, Strand G), TIALLS (SEQ ID NO: 27, Strand G), or TAVVTN (SEQ ID NO: 28, Strand H). In some aspects, the first peptide of the polypeptide probe may bind to strand For strand H (e.g., a strand comprising the peptides AEVVFT (SEQ ID NO: 25) or TAVVTN (SEQ ID NO: 28). In some aspects the first peptide of the polypeptide probe may bind to strand F. In some aspects, the first peptide of the polypeptide probe may bind to strand H. In some aspects, the second peptide of the polypeptide probe may bind a region of the TTR fibril (e.g., a strand or a loop) comprising a peptide selected from VAVHVF (SEQ ID NO: 22, Strand B), IYKVEI (SEQ ID NO: 23, Strand E), KALGIS (SEQ ID NO: 24, loop connecting Strands E and F), AEVVFT, SEQ ID NO: 25; Strand F), YTIAAL, SEQ ID NO: 26, Strand G), TIALLS (SEQ ID NO: 27, Strand G), or TAVVTN (SEQ ID NO: 28, Strand H). In some aspects, the second peptide of the polypeptide probe may bind to strand F or strand H (e.g., a strand comprising the peptides AEVVFT (SEQ ID NO: 25) or TAVVTN (SEQ ID NO: 28)). In some aspects the second peptide of the polypeptide probe may bind to strand F. In some aspects, the second peptide of the polypeptide probe may bind to strand H. In some aspects, the first peptide of the polypeptide probe binds to strand F and the second peptide of the polypeptide probe binds to strand H. In some aspects, the first peptide of the polypeptide probe binds to strand H and the second peptide of the polypeptide probe binds to strand F.

In any of the polypeptide probes described herein, the first peptide and/or the second peptide may comprise HVAHPFVEFTE (SEQ ID NO: 1) and/or SYVTNPTSYAVT (SEQ ID NO: 2). For example, in some aspects, the first peptide may comprise or consist of HVAHPFVEFTE (SEQ ID NO: 1). In some aspects, the first peptide may comprise or consist of SYVTNPTSYAVT (SEQ ID NO: 2). In other aspects, the second peptide may comprise or consist of HVAHPFVEFTE (SEQ ID NO:1). In other aspects, the second peptide may comprise or consist of SYVTNPTSYAVT (SEQ ID NO: 2).

In some aspects, the first peptide comprises or consists of HVAHPFVEFTE (SEQ ID NO: 1) and the second peptide comprises or consists of SYVTNPTSYAVT (SEQ ID NO: 2). In other aspects, the first peptide comprises or consists of SYVTNPTSYAVT (SEQ ID NO: 2) and the second peptide comprises or consists of HVAHPFVEFTE (SEQ ID NO: 1).

### b) Linkers

The first and second peptides of the polypeptide probe may be covalently linked via a flexible peptide linker. Suitable linkers may include a glycine rich linker (e.g., GGGSTE, SEQ ID NO: 3). Other suitable linkers include EAAAK (SEQ ID NO: 4), PAPAP (SEQ ID NO: 4), or GGGGGG (SEQ ID NO: 6). Other suitable flexible linkers are known in the art.

### c) Epitope Tags

In some aspects, the polypeptide probes further comprise one or more epitope tags. Epitope tags may in some aspects be used to facilitate purification and concentration of the polypeptide probe (e.g., off of an affinity column). In other aspects, the epitope tag may increase solubility of the polypeptide probe. In some aspects, epitope tag can comprise an amino acid sequence that increases peptide solubility (e.g., a plurality of arginine residues); and/or an amino acid sequence that facilitates monitoring or manipulation of the peptide (e.g., a plurality of lysine and/or histidine residues). In some aspects, the plurality of arginine, lysine and/or histidine residues comprises 3 to 12 arginine, lysine and/or histidine residues (e.g., 3, 4, 5, 6, 7, 8, 9, 10, 11, or 12 arginine, lysine and/or histidine residues). In some aspects, the epitope tag comprises DYKDDDK (SEQ ID NO: 7) or YPYDVPDYA (SEQ ID NO: 8). In some aspects the epitope tag comprises a plurality of arginine residues (e.g., RRRR, SEQ ID NO: 29). In some aspects, the epitope tag comprises a plurality of histidine residues (e.g., HHHHHH, SEQ ID NO: 30). In some aspects, the epitope tag comprises a plurality of histidine and arginine residues (e.g., HHHHHH-RRRR, SEQ ID NO: 31).

### d) Detectable Labels

In embodiments of the disclosure, the polypeptide probe is detectably labeled. Labeled peptides can be used, e.g., to better understand the mechanism of action and/or the cellular location of the inhibitory peptide. Suitable labels which enable detection (e.g., provide a detectable signal, or can be detected) are conventional and well-known to those of skill in the art. Suitable detectable labels include, e.g., radioactive active agents, fluorescent labels, and the like. Methods for attaching such labels to a protein, or assays for detecting their presence and/or amount, are conventional and well-known.

The polypeptide probe may further comprise one or more detectable labels. Suitable detectable labels that may be conjugated to peptides are known in the art. In non-limiting examples, the detectable label comprises TMR, tetramethylrhodamine (e.g., TAMRA) or Fluorescein isothiocyanate (FITC). In some aspects, a linker is required to connect the label to the peptide. For example, conjugating FITC to a peptide requires an auxiliary linker (e.g., aminohexanoic acid). Therefore, in some aspects, the polypeptide further comprises an aminohexanoic acid linker.

### 2. Exemplary Polypeptide Probes

In accordance with various aspects of the disclosure, the polypeptide probes may comprise the amino acid sequence RRRRHVAHPFVEFTEGGGSTERRRRSYVTNPTSYAVT (SEQ ID NO: 9). In other aspects, the polypeptide probes may comprise the amino acid sequence YPYDVPDYARRRRHVAHPFVEFTEGGGSTERRRRSYVTNPTSYAVT (SEQ ID NO: 10), DYKDDDDKRRRRHVAHPFVEFTEGGGSTERRRRSYVTNPTSYAVT (SEQ ID NO: 11) or HHHHHHRRRRHVAHPFVEFTEGGGSTERRRRSYVTNPTSYAVT (SEQ ID NO: 12). In some aspects, the polypeptide probes may consist of the amino acid sequence YPYDVPDYARRRRHVAHPFVEFTEGGGSTERRRRSYVTNPTSYAVT (SEQ ID NO: 10), DYKDDDDKRRRRHVAHPFVEFTEGGGSTERRRRSYVTNPTSYAVT (SEQ ID NO: 11) or HHHHHHRRRRHVAHPFVEFTEGGGSTERRRRSYVTNPTSYAVT (SEQ ID NO: 12) and a detectable label.

In further aspects of the disclosure, the polypeptide probe is selected from TAMRA-YPYDVPDYA-RRRRHVAHPFVEFTEGGGSTERRRRSYVTNPTSYAVT (SEQ ID NO: 14), TAMRA-DYKDDDDK-RRRRHVAHPFVEFTEGGGSTERRRRSYVTNPTSYAVT (SEQ ID NO: 15), TAMRA-HHHHHH-RRRRHVAHPFVEFTEGGGSTERRRRSYVTNPTSYAVT (SEQ ID NO: 16), FITC-Ahx-YPYDVPDYA-RRRRHVAHPFVEFTEGGGSTERRRRSYVTNPTSYAVT (SEQ ID NO: 17), FITC-Ahx-DYKDDDDK-RRRRHVAHPFVEFTEGGGSTERRRRSYVTNPTSYAVT (SEQ ID NO: 18), FITC-Ahx-HHHHHH-RRRRHVAHPFVEFTEGGGSTERRRRSYVTNPTSYAVT (SEQ ID NO: 19), FITC-Ahx-RRRRHVAHPFVEFTEGGGSTERRRRSYVTNPTSYAVT (SEQ ID NO: 20), wherein TAMRA is tetramethylrhodamine, FITC is Fluorescein isothiocyanate (FITC) and Ahx is an aminohexanoic acid linker.

In further embodiments, the polypeptide probe is selected from FITC-Ahx-HHHHHH-RRRRHVAHPFVEFTEGGGSTERRRRSYVTNPTSYAVT (SEQ ID NO: 19), FITC-Ahx-YPYDVPDYA-RRRRHVAHPFVEFTEGGGSTERRRRSYVTNPTSYAVT (SEQ ID NO: 17), and FITC-Ahx-DYKDDDDK-RRRRHVAHPFVEFTEGGGSTERRRRSYVTNPTSYAVT (SEQ ID NO: 18). For example, in some embodiments, the polypeptide probe comprises FITC-Ahx-HHHHHH-RRRRHVAHPFVEFTEGGGSTERRRRSYVTNPTSYAVT (SEQ ID NO: 19). In some embodiments, the polypeptide probe consists of FITC-Ahx-HHHHHH-RRRRHVAHPFVEFTEGGGSTERRRRSYVTNPTSYAVT (SEQ ID NO: 19), wherein in any of these embodiments FITC is Fluorescein isothiocyanate (FITC) and Ahx is an aminohexanoic acid linker.

In various aspects, the polypeptide has an unexpectedly low detection threshold. As used herein, a “detection threshold” refers to the concentration of the polypeptide that may be used and still register a recoverable signal in the presence of a detectable amount of the target (e.g., TTR fibrils). As shown further in Examples 2 and 16 below, the polypeptide probes used here have a very low detection threshold—with an ability to detect nanograms of target (TTR fibrils) with as low as 1 fM of the probe. Accordingly, in some aspects, the lower limit of the detection concentration is 1-500 fM, 1-100 fM, 1-50 fM, 1-40 fM, 1-30 fM, 1-20 fM, 1-10 fM, or 1-5 fM. In some aspects, a lower limit concentration of the polypeptide that may be used to detect a TTR fibril in a sample is about 1 fM, about 2 fM, about 3 fM, about 4 fM, about 5 fM, about 6 fM, about 7 fM, about 8 fM, about 9 fM, or about 10 fM. In various aspects, the lower limit of a concentration of the polypeptide that may be used to detect a TTR fibril in a sample is about 1 fM.

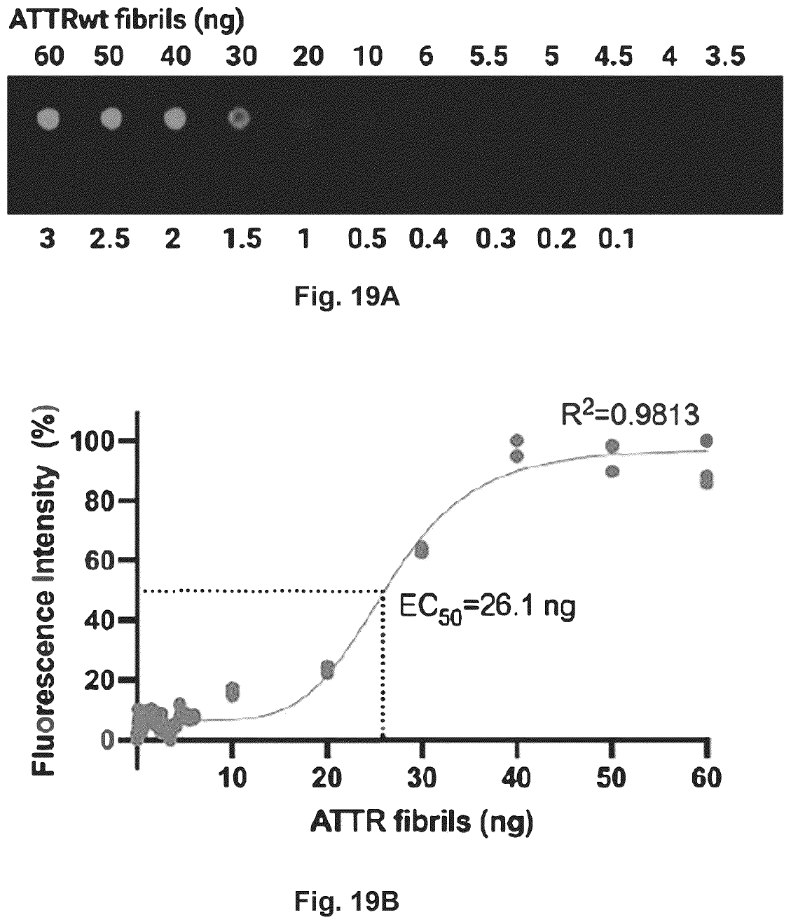

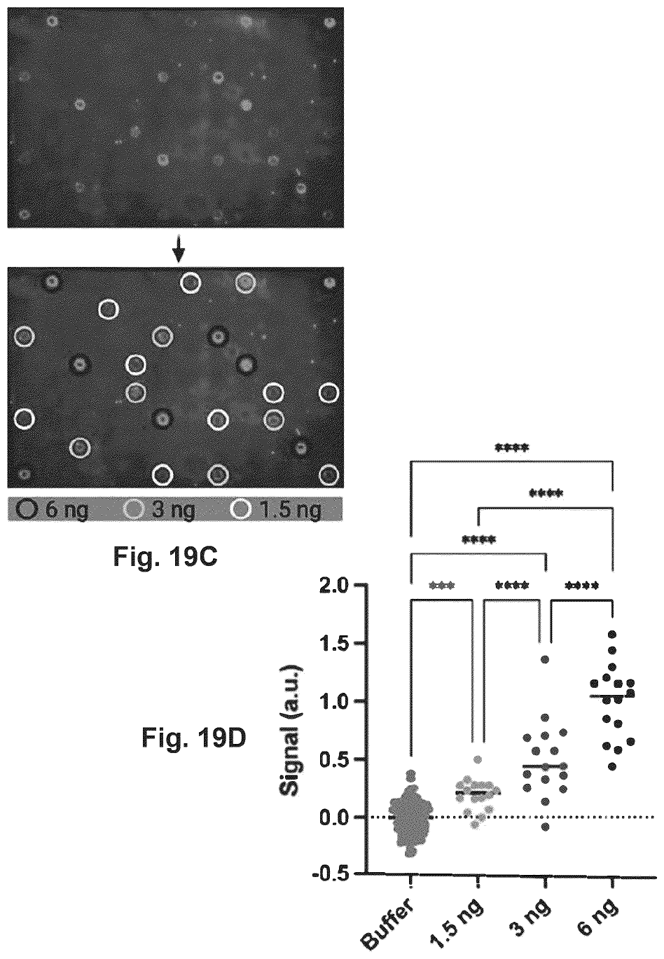

In various aspects, the polypeptide has an unexpectedly low EC50. As used herein, an “EC50” refers to the concentration of the target that elicits a half-maximal signal when detected by a standard concentration of the probe. In some aspects, the polypeptide probe has an EC50 of less than 1 μg of target. In some aspects, the polypeptide probe has an EC50 of about 1 to 100 ng of target. In some aspects, the polypeptide probe has an EC50 of about 10 to 50 ng, about 10 to 40 ng, about 10 to 30 ng, or about 20 to 30 ng of the target. In some aspects, the polypeptide probe may have an EC50 of about 26.1 ng of the target. Methods of determining an EC50 for a probe provided herein are within the ordinary skill in the art and are specifically described further in the Examples below (see FIG. 19B and Example 16 below).

### 3. Variants and Modifications

In further aspects, the polypeptide probe may further comprise one or more non-natural amino acids or modifications. In other aspects, the polypeptide probe may not comprise any non-natural amino acids or other modifications.

Amino acid substitutions. In some embodiments, amino acids other than the ones noted above in the consensus sequence are substituted. These amino acids can help protect the peptides against proteolysis or otherwise stabilize the peptides, and/or contribute to desirable pharmacodynamic properties in other ways. In some embodiments, the non-natural amino acids allow the peptide to bind more tightly to the target because the side chains optimize hydrogen bonding and/or apolar interactions with it. In addition, non-natural amino acids offer the opportunity of introducing detectable markers, such as strongly fluorescent markers which can be used, e.g., to measure values such as inhibition constants. Also included are peptide mimetics, such as, e.g., peptoids, beta amino acids, N-ethylated amino acids, and small molecule mimetics.

Non-Natural Amino Acid Substitutions—In one embodiment, non-natural amino acids are substituted for amino acids in the sequence. More than 100 non-natural amino acids are commercially available. These include, for example the non-natural amino acids listed in the tables below.

Non-Natural Amino Acids which can Substitute for LEU:

| | |
|---|---|
| Fmoc-L-cyclohexylglycine | CAS No.: 161321-36-4 |
| Fmoc-L-phenylglycine | CAS No.: 102410-65-1 |
| Fmoc-4-hydroxy-D-phenylglycine | CAS No.: 178119-93-2 |
| Fmoc-L-a-t-butylglycine | CAS No.: 132684-60-7 |
| Fmoc-cyclopentyl-Gly-OH | CAS No.: 220497-61-0 |
| Fmoc-L-2-indanylglycine | CAS No.: 205526-39-2 |

Non-Natural Amino Acids which can Substitute for THR:

| | |
|---|---|
| Fmoc-Thr(tBu)-OH | CAS No.: 71989-35 |
| Fmoc-(RS)-2-amino-3-hydroxy-3- | CAS No.: 105504-72-1 |
| methylbutanoic acid | |
| Boc-N-Me-allo-Ile-OH | CAS No.: 136092-80-3 |
| Fmoc-Homoleu-OH | CAS No.: 180414-94-2 |

Non-Natural Amino Acids which can Substitute for ARG:

| | |
|---|---|
| Fmoc-Nw-nitro-L-arginine | CAS No.: 58111-94-7 |
| Fmoc-L-citrulline | CAS No.: 133174-15-9 |

Non-Natural Amino Acids which can Substitute for TYR:

| | |
|---|---|
| Fmoc-3-amino-L-tyrosine | CAS No.: 726181-70-0 |
| Fmoc-3-nitro-L-tyrosine | CAS No.: 136590-09-5 |
| Fmoc-3-methoxy-L-tyrosine | CAS No.: 881911-32-6 |
| Fmoc-3-iodo-L-tyrosine | CAS No.: 134486-00-3 |
| Fmoc-3-chloro-L-tyrosine | CAS No.: 478183-58-3 |
| Fmoc-3,5-dibrimo-L-tyrosine | CAS No.: 201484-26-6 |

Non-Natural Amino Acids which can Substitute for LYS:

| | |
|---|---|
| Fmoc-Lys(retro-Abz-Boc)-OH | CAS No.: 159322-59-5 |
| Fmoc-Lys(Mca)-OH | CAS No.: 386213-32-7 |
| Fmoc-(No-4-methyltrityl)-L- ornithine | CAS No.: 343770-23-0 |
| N-a-Fmoc-N-£-(d-Biotin)-L-lysine | CAS No.: 146987-10-2 |

In another embodiment, one or more (e.g., 1, 2, 3, 4, 5, 6, 7, 8, 9, 10, 11, 12, 13, 14, 15, 16 or 17) N-methylated residues are included in the peptide. For example, in some aspects the polypeptide probe may further comprise an N-methyl group. In other aspects, the polypeptide probe does not comprise an N methyl group. A variety of other types of active variants are encompassed.

A polypeptide probe of the disclosure can comprise, e.g., L-amino acids, D-amino acids, non-natural amino acids, or combinations thereof. Active variants include molecules comprising various tags at the N-terminus or the C-terminus of the peptide. For example, a polypeptide of the instant disclosure may comprise as tags as its N-terminus and/or at its C-terminus: 1, 2, 3, 4, 5, 6, 7, 8, 9 or 10 Lysine residues; 1, 2, 3, 4, 5, 6, 7, 8, 9 or 10 Arginine residues; 1, 2, 3, 4, 5, 6, 7, 8, 9 or 10 Glutamate residues; 1, 2, 3, 4, 5, 6, 7, 8, 9 or 10 Aspartate residues; combinations of these amino acid residues; or other polar tags that will be evident to a skilled worker. In some aspects, the tags may be provided within the peptide. Other active variants include mutations of the sequences of the polypeptide probe sequence which increase affinity of the inhibitory peptides for the TTR molecule (oligomer, fibril, etc.).

In one embodiment of the disclosure, a polypeptide probe described herein is isolated or purified, using conventional techniques such as the methods described herein. By “isolated” is meant separated from components with which it is normally associated, e.g., components present after the peptide probe is synthesized. An isolated polypeptide probe can be a cleavage product of a protein which contains the polypeptide sequence. A “purified” polypeptide probe can be, e.g., greater than 80%, 85%, 90%, 95%, 98% or 99% pure. As a polypeptide probe described herein comprises a detectable label, the polypeptide portion of the probe may be synthesized and then conjugated to the detectable label. In some aspects, the pharmaceutical composition may comprise a polypeptide probe lacking the detectable label, as long as at least one polypeptide probe in the composition comprises the detectable label. In some aspects, the composition is provided comprising the labeled polypeptide probe and, optionally, the unlabeled polypeptide probe, wherein at least 50%, at least 60%, at least 70%, at least 80%, at least 90%, at least 95%, at least 96%, at least 97%, at least 98%, at least 99% or 100% of the polypeptide probe is conjugated to the detectable label.

A polypeptide probe of the disclosure can be synthesized (e.g., chemically or by recombinant expression in a suitable host cell) by any of a variety of art-recognized methods. In order to generate sufficient quantities of an inhibitory peptide for use in a method of the disclosure, a practitioner can, for example, using conventional techniques, generate nucleic acid (e.g., DNA) encoding the peptide and insert it into an expression vector, in which the sequence is under the control of an expression control sequence such as a promoter or an enhancer, which can then direct the synthesis of the peptide. For example, one can (a) synthesize the DNA de novo, with suitable linkers at the ends to clone it into the vector; (b) clone the entire DNA sequence into the vector; or (c) starting with overlapping oligonucleotides, join them by conventional PCR-based gene synthesis methods and insert the resulting DNA into the vector. Suitable expression vectors (e.g., plasmid vectors, viral, including phage, vectors, artificial vectors, yeast vectors, eukaryiotic vectors, etc.) will be evident to skilled workers, as will methods for making the vectors, inserting sequences of interest, expressing the proteins encoded by the nucleic acid, and isolating or purifying the expressed proteins.

### 4. Pharmaceutical Compositions

Any polypeptide probes disclosed herein may be formulated into a pharmaceutical composition. In some embodiments, pharmaceutical composition may further include one or more pharmaceutically acceptable carriers, diluents or excipients. Any of the pharmaceutical compositions to be used in the present methods can comprise pharmaceutically acceptable carriers, excipients, or stabilizers in the form of lyophilized formations or aqueous solutions.

The carrier in the pharmaceutical composition must be “acceptable” in the sense that it is compatible with the active ingredient of the composition, and preferably, capable of stabilizing the active ingredient and not deleterious to the subject to be treated. For example, “pharmaceutically acceptable” may refer to molecular entities and other ingredients of compositions comprising such that are physiologically tolerable and do not typically produce untoward reactions when administered to a mammal (e.g., a human). In some examples, the “pharmaceutically acceptable” carrier used in the pharmaceutical compositions disclosed herein may be those approved by a regulatory agency of the Federal or a state government or listed in the U.S. Pharmacopeia or other generally recognized pharmacopeia for use in mammals, and more particularly in humans.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides;
disaccharides; and other carbohydrates; metal complexes; and/or non-ionic surfactants. See, e.g. Remington: The Science and Practice of Pharmacy 20th Ed. (2000) Lippincott Williams and Wilkins, Ed. K. E. Hoover.

The pharmaceutical compositions disclosed herein may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycols.

### III. Methods

In various aspects of the present disclosure, the polypeptide probes provided herein may be used in methods to detect transthyretin oligomers, fibrils or molecules in a subject in need thereof. An advantage of the methods herein is the ability of the polypeptide probes to detect aggregated forms of TTR (e.g., oligomers and fibrils), which allows for earlier detection and therefore, earlier diagnosis of TTR related conditions along with earlier and more accurate risk determination for future TTR aggregation, and improved therapeutic monitoring. In particular, the polypeptide probes may allow for earlier detection and diagnosis of preeclampsia. Therefore, methods of detecting transthyretin oligomers, fibrils, or molecules in a subject are provided herein below as well as methods of applying this detection to diagnose, evaluate risk, monitor therapies, or treat an individual having or suspected of having preeclampsia.

In some aspects, the present disclosure is directed to a method of detecting an oligomer, aggregate or fibril of transthyretin in a sample, the method comprising (a) contacting the sample with a polypeptide probe described herein, (b) allowing the polypeptide probe to bind any oligomers, aggregates, or fibrils of transthyretin in the sample, and (c) detecting a complex comprising the polypeptide and an oligomer, aggregate or fibril of transthyretin, wherein the presence of the complex correlates to the presence of an oligomer, aggregate or fibril of transthyretin in the sample.

Another aspect of the disclosure is a method of diagnosing a subject with a disease or condition which is mediated by the presence of fibrillated or aggregated TTR (sometimes referred to herein as a TTR-mediated disease or condition). In some aspects, the method comprises (a) detecting a transthyretin oligomer, fibril or molecule in a sample obtained from the subject according to methods provided herein using a polypeptide probe of the disclosure, and (b) diagnosing a subject with a TTR mediated disease or condition if the transthyretin oligomer, fibril or molecule is detected in the sample. In some aspects, the subject is diagnosed with the TTR mediated disease or condition if levels of the transthyretin oligomers, fibrils or molecules detected in the sample exceed a threshold.

Another aspect of the disclosure is a method of diagnosing a subject with preeclampsia. In some aspects, the method comprises (a) detecting a transthyretin oligomer, fibril or molecule in a sample obtained from the subject according to methods provided herein using a polypeptide probe of the disclosure, and (b) diagnosing a subject with preeclampsia if the transthyretin oligomer, fibril or molecule is detected in the sample. In some aspects, the method comprises (a) detecting a transthyretin oligomer, fibril or molecule in a sample obtained from the subject according to methods provided herein using a polypeptide probe of the disclosure, and (b) diagnosing a subject with preeclampsia if the transthyretin oligomer, fibril or molecule is detected in the sample. In some aspects, the subject is diagnosed with preeclampsia if levels of the transthyretin oligomers, fibrils or molecules detected in the sample exceed a threshold.

Another aspect of the disclosure is a method of determining whether a subject is at risk of TTR aggregation (e.g., a screening method). In various aspects, the method can comprise (a) detecting transthyretin oligomer, fibril or molecule in a sample obtained from the subject according to methods provided herein using a polypeptide probe of the disclosure, and (b) identifying the subject as at risk for TTR aggregation if the transthyretin oligomer, fibril or molecule is detected in the sample. In some aspects, the subject is identified at risk for TTR aggregation if levels of transthyretin oligomers, fibrils, and/or molecules exceed a threshold.

Another aspect of the disclosure is a method of determining whether a subject is at risk of preeclampsia (e.g., a screening method). In various aspects, the method can comprise (a) detecting transthyretin oligomer, fibril or molecule in a sample obtained from the subject according to methods provided herein using a polypeptide probe of the disclosure, and (b) identifying the subject as at risk for preeclampsia if the transthyretin oligomer, fibril or molecule is detected in the sample. In some aspects, the subject is identified at risk for preeclampsia if levels of transthyretin oligomers, fibrils, and/or molecules exceed a threshold.

Another aspect of the disclosure is a method for monitoring the efficacy of a therapy administered to a subject to treat a TTR-mediated disease or condition. In some aspects, the method comprises (a) detecting a transthyretin oligomer, fibril or molecule in a first sample obtained from the subject according to methods provided herein using a polypeptide probe of the disclosure, (b) administering a therapeutic to the subject, and (c) detecting a transthyretin oligomer, fibril or molecule in a second sample obtained from the subject after administration of the therapeutic, and (d) monitoring the efficacy of the therapeutic by comparing the level of detection of aggregated or fibrillar transthyretin in the first sample with the second sample. In various aspects, the therapeutic is determined to be effective if fewer oligomers, aggregates and/or fibrils of transthyretin are detected in the second sample compared to the first sample. In further aspects, the subject has been diagnosed with the TTR-mediated disease or condition using a method provided herein (e.g., with the polypeptide probes). In some aspects, the method further comprises determining an effective amount of a therapeutic by monitoring efficacy of different doses of the therapeutic. Suitable therapeutics that may be tested are described below.

Another aspect of the disclosure is a method for monitoring the efficacy of a therapy administered to a subject to treat preeclampsia. In some aspects, the method comprises (a) detecting a transthyretin oligomer, fibril or molecule in a first sample obtained from the subject according to methods provided herein using a polypeptide probe of the disclosure, (b) administering a therapeutic to the subject, and (c) detecting a transthyretin oligomer, fibril or molecule in a second sample obtained from the subject after administration of the therapeutic, and (d) monitoring the efficacy of the therapeutic by comparing the level of detection of aggregated or fibrillar transthyretin in the first sample with the second sample. In various aspects, the therapeutic is determined to be effective if fewer oligomers, aggregates and/or fibrils of transthyretin are detected in the second sample compared to the first sample. In further aspects, the subject has been diagnosed with preeclampsia using a method provided herein (e.g., with the polypeptide probes). In some aspects, the method further comprises determining an effective amount of a therapeutic by monitoring efficacy of different doses of the therapeutic. Suitable therapeutics that may be tested are described below.

Another aspect of the disclosure is a method of treating a subject with an effective amount of a therapeutic for a TTR-mediated disease or condition. In some aspects the method comprises determining an effective amount of a therapeutic for a TTR-mediated disease or condition in a subject using the method of determining efficacy of a treatment provided herein, and then administering the effective amount of the therapeutic to the subject. In some aspects, the method comprises diagnosing the subject with the TTR mediated disease or condition, as described herein, and then administering a therapeutic to the subject. In some aspects, the method comprises determining that a subject is at risk for TTR aggregations according to a method described herein and then administering a therapeutic to the subject. Suitable therapeutics are described below.

Another aspect of the disclosure is a method of treating a subject with an effective amount of a therapeutic for preeclampsia. In some aspects the method comprises determining an effective amount of a therapeutic for preeclampsia in a subject using the method of determining efficacy of a treatment provided herein, and then administering the effective amount of the therapeutic to the subject. In some aspects, the method comprises diagnosing the subject with preeclampsia, as described herein, and then administering a therapeutic to the subject. In some aspects, the method comprises determining that a subject is at risk for preeclampsia according to a method described herein and then administering a therapeutic to the subject. Suitable therapeutics are described below.

In any of the preceding methods, the sample(s) can be obtained from a subject who is pregnant. In any of the preceding methods, the sample(s) can be obtained from a subject at risk of developing preeclampsia or another pregnancy complication. In any of the preceding methods, the sample(s) can be obtained from a subject having or suspected of having preeclampsia. In various aspects, the methods herein may comprise diagnosing a subject with preeclampsia based on levels of aggregated or oligomeric TTR in a patient sample. In various aspects, the sample may comprise a blood, serum, or plasma sample. In various aspects, the sample may comprise a placental lysate. In various aspects, levels of aggregated TTR may be quantified or assessed using the probes of the instant disclosure.

In various aspects, the methods herein may comprise administering a suitable therapeutic to a subject. In various aspects, the suitable therapeutic can be an inhibitor of transthyretin expression and/or aggregation. In various aspects, the therapeutic comprises an inhibitor of TTR expression. In some aspects, the therapeutic comprises an inhibitor of TTR aggregation. Suitable inhibitors of TTR expression can include, for example, gene silencers which are agents that reduce or “silence” gene expression. Suitable gene silencers are known in the art and can include, for example, interfering RNAs (e.g., RNAi) and antisense oligonucleotides (ASOs). Suitable inhibitors of TTR aggregation can include any agent (e.g., a small molecule or an antibody) that binds to and stabilizes the conformation of a TTR monomer or fibril to prevent association and aggregation. In some aspects, the therapeutic comprises tafamidis. In some aspects, the therapeutic comprises an anti-TTR antibody or a gene silencer (e.g., siRNA or antisense oligonucleotide targeting the TTR gene or TTR mRNA).

In any of the foregoing aspects, a TTR related disorder or disease may include preeclampsia.

In any of the methods described herein, the subject may be mammalian. In any aspect described herein, the subject may be human.

Having described several embodiments, it will be recognized by those skilled in the art at various modifications, alternative constructions, and equivalents may be used without departing from the spirit of the present disclosure. Additionally, a number of well-known processes and elements have not been described in order to avoid unnecessarily obscuring the present disclosure. Accordingly, this description should not be taken as limiting the scope of the present disclosure.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Introduction to Examples

ATTR amyloidosis (transthyretin amyloidosis) is a highly underdiagnosed fatal disease caused by the systemic deposition of amyloid fibrils composed of transthyretin (TTR when functional, or ATTR when in its amyloidogenic form). ATTR deposition is triggered by the destabilization of the functional tetrameric form of TTR by aging in wild-type ATTR (ATTRwt) amyloidosis or a mutation in variant ATTR (ATTRv) amyloidosis. The clinical presentation of ATTRwt amyloidosis is relatively homogeneous, characterized with primarily cardiomyopathy and usually impacting men late in life. ATTRv amyloidosis is often variable and unpredictable, and may involve polyneuropathy, carpal tunnel syndrome, gastrointestinal and eye involvement, and/or cardiomyopathy. Some studies estimate the presence of pathogenic ATTRwt deposits in cardiac tissues of approximately 25% of individuals over 80 years of age. The most common mutations in the United States are ATTR-V30M, ATTR-T60A, and ATTR-V1221. Of note, the ATTR-V1221 mutation is estimated to impact 3-4% of the African American population, making them more susceptible to developing ATTRv amyloidosis and heart failure. Despite this, and probably because of its heterogeneous clinical presentation, this disease is underdiagnosed or misdiagnosed. These difficulties are amplified by the lack of robust tools that can detect the disease regardless of patient phenotype.

The diagnostic process for ATTR amyloidosis is costly and complex, resulting in delayed patient diagnosis and ultimately poorer clinical prognosis due to delayed start of treatments. After an individual is suspected of having amyloidosis, which may take years of tests, the presence of amyloid should first be confirmed. Confirming amyloid deposition usually involves staining a biopsy section with Congo red dye. This dye has variable affinity and is not specific for ATTR, since it can bind amyloid deposits from other precursor proteins. One of the largest obstacles in the diagnostic process is distinguishing between ATTR amyloidosis and other forms of systemic amyloidosis such as light-chain amyloidosis. Thus, if Congo red confirms the presence of amyloid, mass spectrometry or antibody-based assays is used to identify the protein forming the amyloid. However, mass spectrometry may require expensive equipment, highly trained individuals to evaluate the data, and the results may be inconclusive. Many commercial antibodies are unreliable in a diagnostic setting. In recent years, radiolabeled tracers such as 99mTc-PYP have become more common in detecting ATTR amyloid. Radiolabeled tracers are suitable only for cardiac cases of ATTR amyloidosis and they bind calcifications in the heart rather than the amyloid. This technique requires the injection of a radioactive reagent, with potential health risks. Once the amyloid is confirmed to be ATTR, clinicians may genotype the patient to determine whether they have a mutation in their TTR protein. In summary, there is no single, robust, unexpensive, safe, and/or high throughput tool that can diagnose ATTR amyloidosis. The development of a biomarker for early diagnosis would ease the massive burden not only on the resources of healthcare systems, but also on the patients suffering from this disease and their families.

Novel biomarkers represent a potential tool for the diagnosis of ATTR amyloidosis. Blood biomarkers that indicate cardiac injury such as natriuretic peptides and cardiac troponins are used to assess ATTR amyloidosis severity and progression, but they are not specific for ATTR amyloidosis. Circulating transthyretin and its ligand retinol binding protein 4 have also been assessed as biomarkers of ATTR amyloidosis, but whether they can be used for diagnosis is yet to be determined. Recently, a peptide probe that binds nonnative ATTR species present in the plasma of polyneuropathie ATTRv patients has been developed along with an immunoassay using an antibody that specifically recognizes these nonnative oligomeric ATTR species in plasma of only ATTR neuropathic patients. More tools are needed to specifically detect ATTR in patients regardless of their genotype or phenotype.

In the following examples, a first-generation transthyretin aggregation detection (TAD1) probe is described for the specific detection of ATTR fibrils and aggregates in patient tissues. This probe displays high specificity for ATTR fibrils in cardiac tissues, recognizes ATTR fibrils in a conformation-dependent manner, and can be used as a tool for detecting ATTR aggregates in plasma from patients with various genotypes and phenotypes. This novel tool reveals the presence of aggregates in blood and may represent a potential screening method for the specific detection of ATTR amyloidosis.

### Example 1: Materials and Methods for Examples 2-10

Dot blot of Extracted Fibrils/Patient Lysate The ability of peptides to recognize TTR species was done using dot blot analysis as described by Saelices et al (Saelices et al. Uncovering the mechanism of aggregation of human transthyretin. J. Biol. Chem. 2015. 27; 290(48):28932-43). Briefly, 0.5 μg of fibrils extracted from ATTR patient hearts, lysates or protein were dotted onto a nitrocellulose membrane (0.2 μM, Bio-Rad). The membrane was blocked for 30 minutes in Ix BSA/TBST. After washing, samples were then probed with the peptides in BSA/TBST for 1 hour. The fluorescence intensity was measured in an Azure Biosystems C600 imaging system through excitation of the membrane at 472 nm and reading emission at 513 nm. Analysis of fluorescence intensity was done using the software program GraphPad Prism.

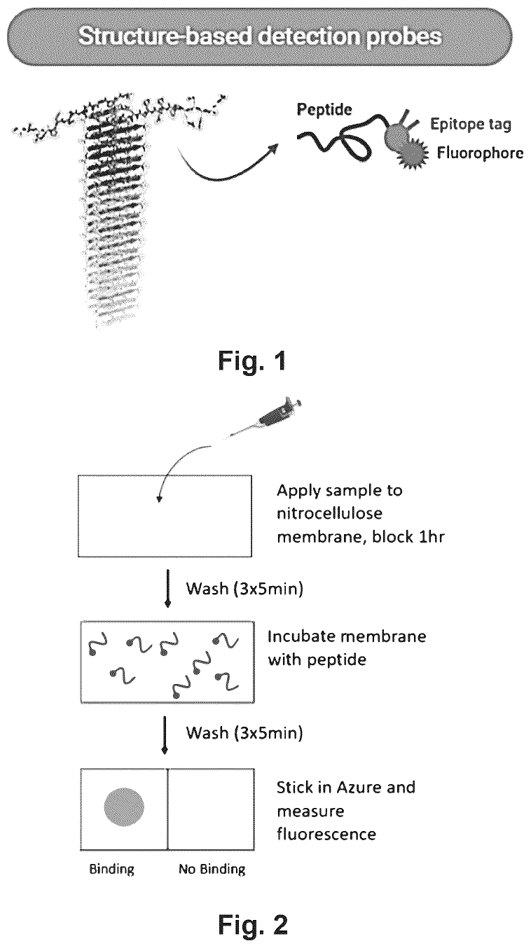

Dot blot of Blood Samples The ability of peptides to recognize TTR species was done using dot blot analysis as described by Saelices et al, 2015 (provided above) and as diagrammed in FIG. 2. Briefly, the specified volume of patient or healthy blood was dotted onto a nitrocellulose membrane (0.2 μM, Bio-Rad). The membrane was blocked for 30 minutes in 1×BSA/TBST. After washing, samples were then probed with the peptides in BSA/TBST for 1 hour. The fluorescence intensity was measured in an Azure Biosystems C600 imaging system through excitation of the membrane at 472 nm and reading emission at 513 nm. Analysis of fluorescence intensity was done using the software program GraphPad Prism.

Immunogold Labeling/Negative Stain Transmission Electron Microscopy. For immunogold labeling, 5 μM peptide was mixed with either 0.25 μMor 0.05 μM Ni-NTA Nanogold beads (Nanoprobes) overnight in binding buffer (20 mM Tris pH 7.6, 150 mM NaCl, 5 mM imidazole). The next morning the supernatant containing unbound nanogold beads was removed. 1 μg/ml of fibrils extracted from a wild-type ATTR patient was spiked into the pellet and the solution was loaded onto a glow-discharged carbon coated EM grid (Electron Microscopy Sciences). The solution was incubated for two minutes before using filter paper to remove excess liquid. The grid was stained with 1% uranyl acetate for one minute. The grid was blotted again to remove excess stain and then visualized using a Tecnai Spirit electron microscope. The ability of peptides to recognize TTR species was done using dot blot analysis as described by Saelices et al, 2015. Briefly, 0.5 μg of fibrils extracted from ATTR patient hearts, lysates or protein were dotted onto a nitrocellulose membrane (0.2 μM, Bio-Rad). The membrane was blocked for 30 minutes in Ix BSA/TBST to prevent nonspecific binding of the probe. After washing, samples were then probed with the peptides in BSA/TBST for 1 hour. The fluorescence intensity was measured in an Azure Biosystems C600 imaging system through excitation of the membrane at 472 nm and reading emission at 513 nm.

Peptide Generation. Peptides were synthesized by LifeTein and sent to use in lyophilized form. The peptide probe used in Examples 2-10 had the sequence: “FITC”-“Ahx”-HHHHHH-RRRRHVAHPFVEFTEGGGSTERRRRSYVTNPTSYAVT (SEQ ID NO: 12), where “FITC” is fluorescein isothiocyanate and “Ahx” is aminohexanoic acid. Prior to running experiments, the peptide was reconstituted in distilled water to yield a final concentration of 5 mM. The resuspended peptide was filtered through a 0.22 μm filter tube by spinning at 20,000 g for 5 minutes at 4° C. to remove any aggregates from the solution.

### Example 2: Peptide Probes Selectively Label ATTR Target Fibrils

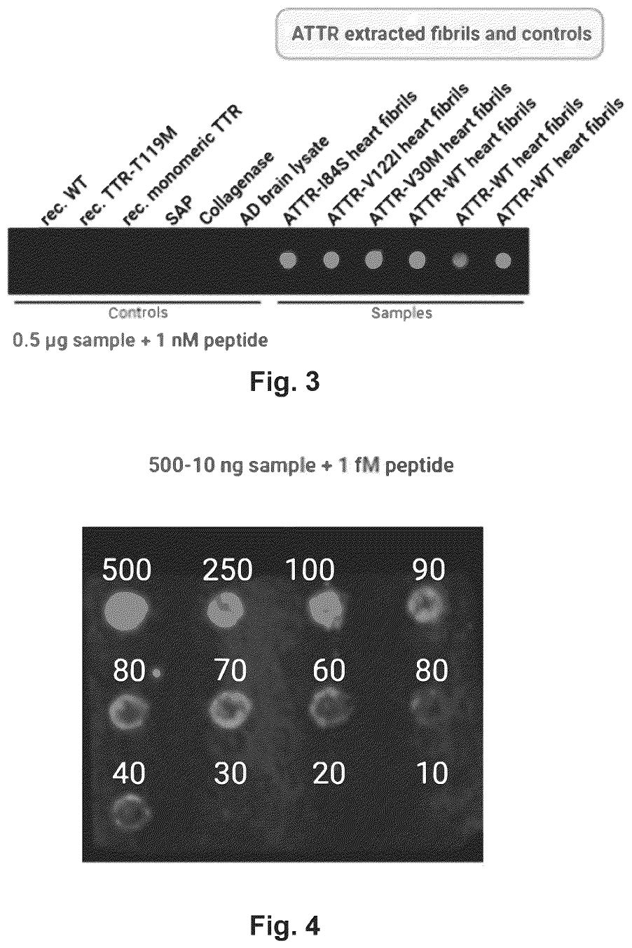

A dot blot was performed on ATTR fibrils extracted from six patients (three with wild type TTR genotype, three with TTR mutations), as well as the following controls:: Wild type tetrameric TTR (Rec WT), Tetrameric TTR with a mutation (T119M) that stabilizes the tetramer (Rec TTR-T119M), Rec Monomeric TTR, serum amyloid protein (SAP, a protein that is reported to associate with ATTR amyloid fibrils), Collagenase (an enzyme used to digest collagen when extracting ATTR fibrils from heart and brain lysate), and extracted tissue from an Alzheimer's Disease patient, which is confirmed to contain amyloid, used as a negative control to show specificity for ATTR fibrils. FIG. 3 shows how the tested probe (at nM concentrations) was specific for ATTR fibrils and not the native protein or other fibrils.

To determine the lower detection limit of the polypeptide probes, different amounts of 184S fibrils (e.g., 10-500 ng) were loaded onto a nitrocellulose membrane and probed with 1fM peptide. FIG. 4 shows that the probes can detect 40 ng of fibrils loaded onto the membrane which is indicative of a significantly lower detection level than expected.

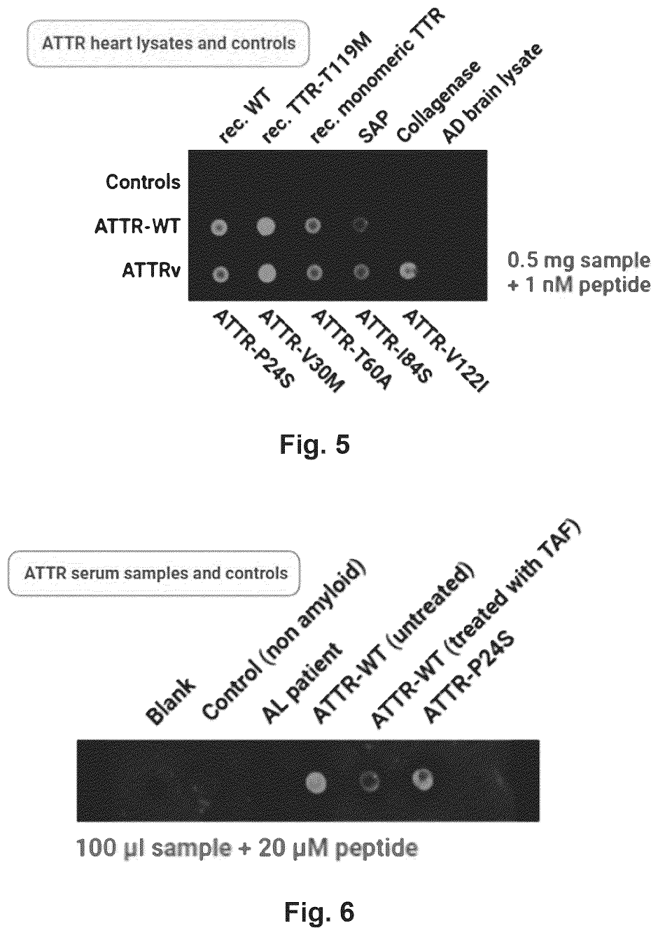

In another experiment, dot blotting was performed with the same control samples used in FIG. 3 along with crude heart lysate from the same patients (4 wild type ATTR patients, second row; followed by 5 mutant ATTR patients, third row). FIG. 5 shows that our probes recognize not only purified fibrils (e.g., FIG. 3), but also fibrils within crude tissue preparations from both WT and mutant ATTR patients.

In another experiment, dot blotting was performed on five serum samples to determine whether probes can recognize ATTR fibrils or aggregates within serum. These samples include a non-amyloid control, a light chain (AL) amyloidosis patient, and three ATTR amyloidosis patients (two wild type TTR, one with a mutation). The results shown in FIG. 6 demonstrate that the probes can detect fibrils or larger aggregates within serum, and that a reduction in signal may be detected after administration of a treatment (e.g., tafamidis, which a current treatments for ATTR amyloidosis), meaning that these probes have potential to measure change in pathogenic ATTR species during the duration of a treatment course. This data supports a surprising and unexpected use for these probes—that they may be used to measure disease outcomes and enable more effective therapeutics.

### Example 3: Peptide Probes Bind ATTR Fibrils

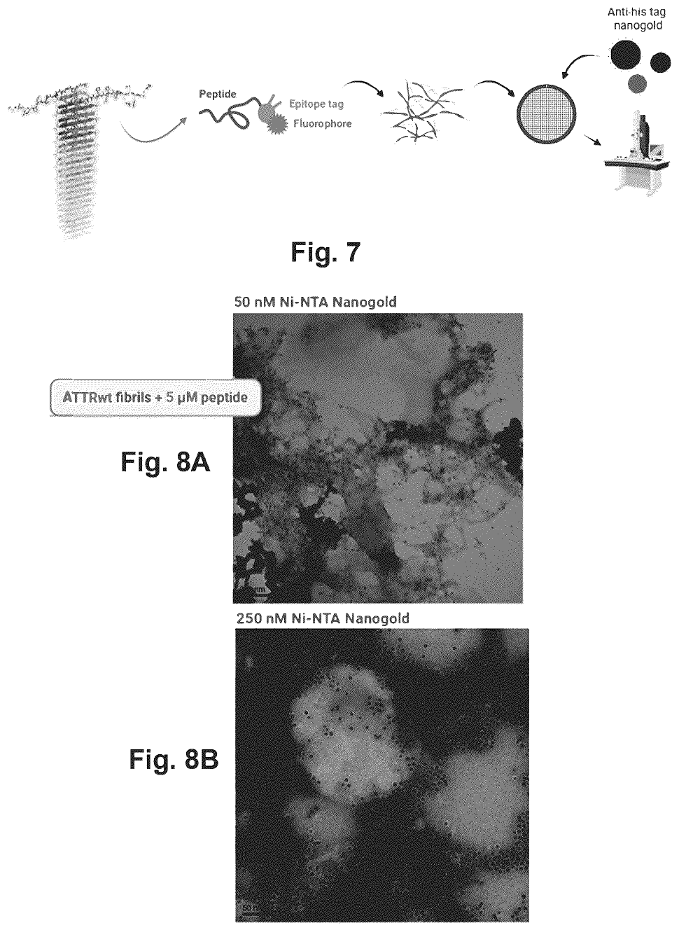

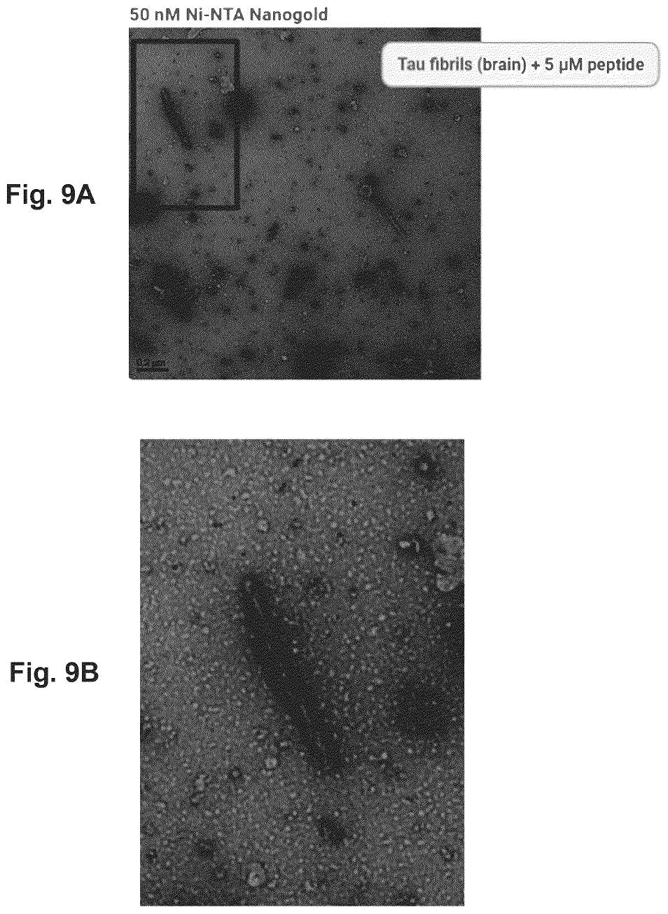

The ability of the peptide probes to bind ATTR fibrils was tested using Immunogold Labeling, as described in Example 1. FIG. 7 shows an illustrative schematic of the experimental protocol used. FIG. 8A and FIG. 8B show labeling and binding of 5 μM peptide to ATTR wildtype fibrils in the presence of 50 nM Ni-NTA Nanogold (FIG. 8A) or 250 nM Ni-NTA Nanogold (FIG. 8B). The labeled peptides localize to extracted ATTR fibrils. In contrast, no labeling was detected between the peptides and tau fibrils as shown in FIG. 9A and FIG. 9B. These results show that these peptides are specific for ATTR fibrils and not other types of amyloids.

### Example 4: Peptide Probes are Specific for Conformation of ATTR Fibrils

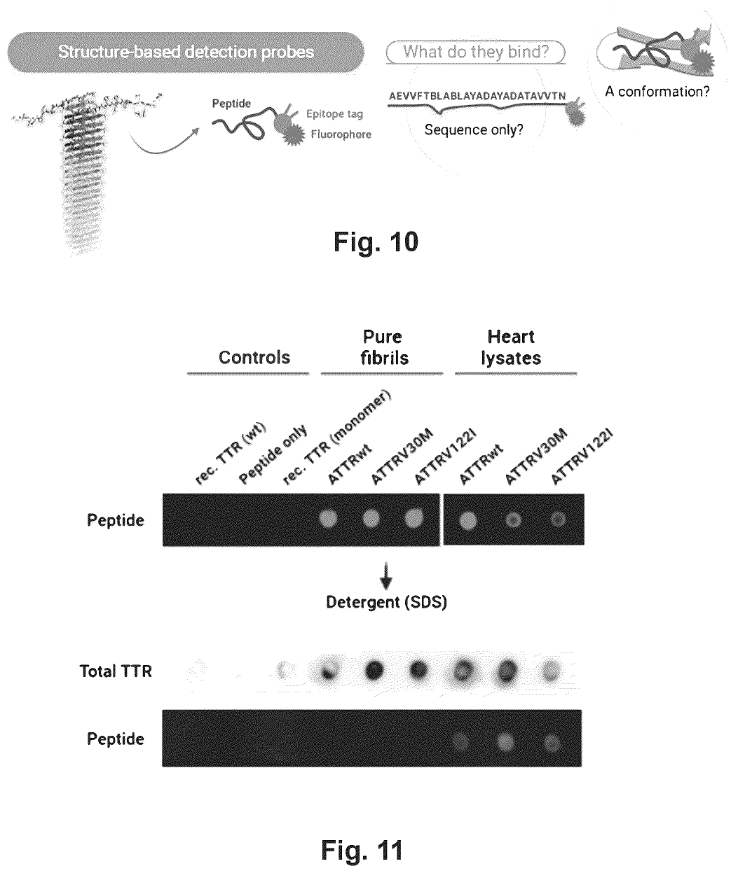

To determine whether the peptide probes had a specific affinity for the TTR sequence or the conformation of the TTR fibril (FIG. 10), another experiment was conducted wherein a detergent (SOS) was applied to the ATTR fibrils prior to the immunoblot analysis. We conducted dot blotting of fibrils extracted from ATTR amyloidosis patients and crude heart lysates from the same patients. We then subjected these samples to denaturing conditions by addition of SOS and heating and did another dot blot probing for total transthyretin as well as fibrils. As shown in FIG. 11, application of the detergent abolished binding to pure fibril samples and significantly reduced binding to heart lysates. This suggests that the peptide probes are structurally selective for ATTR fibrils and show little off target binding to natively folded TTR or unfolded TTR peptides. Together, these data indicate that these peptide probes work in a conformation-dependent manner, rather than a sequence-dependent manner.

### Example 5: Peptide Probes can Bind ATTR Aggregate Species in Plasma

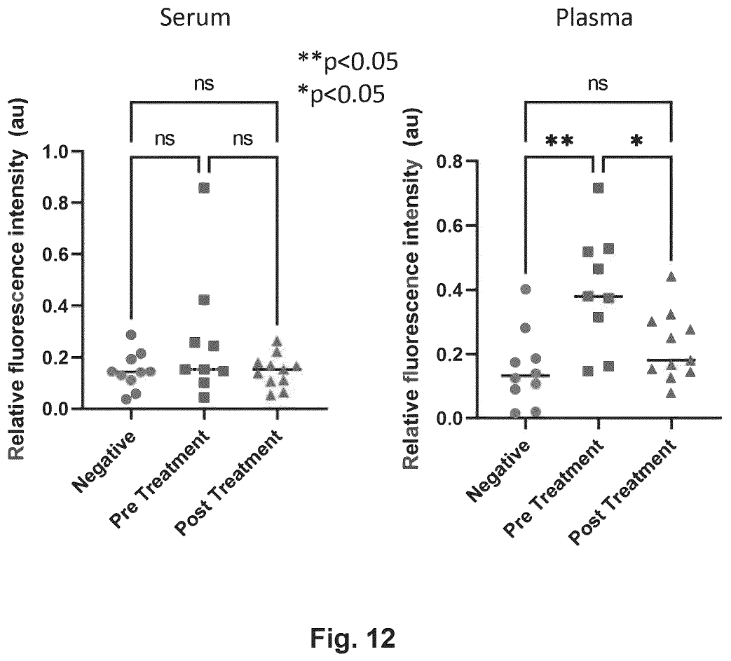

An experiment was conducted to evaluate the specificity of the probe in serum vs. plasma samples. The binding of peptides to ATTR aggregate species was evaluated using dot blot analysis as previously described (Saelices et al., J Biol Chem. 2015 Nov. 27; 290(48):28932-43). Briefly, serum or plasma was obtained from a cohort consisting of either ATTR amyloidosis patients before treatment, ATTR amyloidosis patients after treatment, or healthy controls. 30 μL of each sample was dotted onto a nitrocellulose membrane (0.2 μm, Bio-Rad). The membrane was blocked for 30 minutes in 1× bovine serum albumin (BSA) in tris-buffered saline, 0.1% Tween-20 (TBST). After washing, samples were probed with 15 μM of TAO1 in 1×BSA/TBST for 1 hour. The fluorescence intensity of peptide binding was measured in an Azure Biosystems C600 imaging system through excitation of the membrane at 472 nm and reading emission at 513 nm. Fluorescence intensity of binding was quantified through Image J and analyzed using Prism software. The results show three technical replicates. FIG. 12 shows that the probes successfully labeled plasma, but not serum, samples and may be used to diagnose ATTR amyloidosis and monitor treatment response in patients.

### Example 6: Peptide Probes can Bind ATTR Fibrils in Isolated Tissue Samples

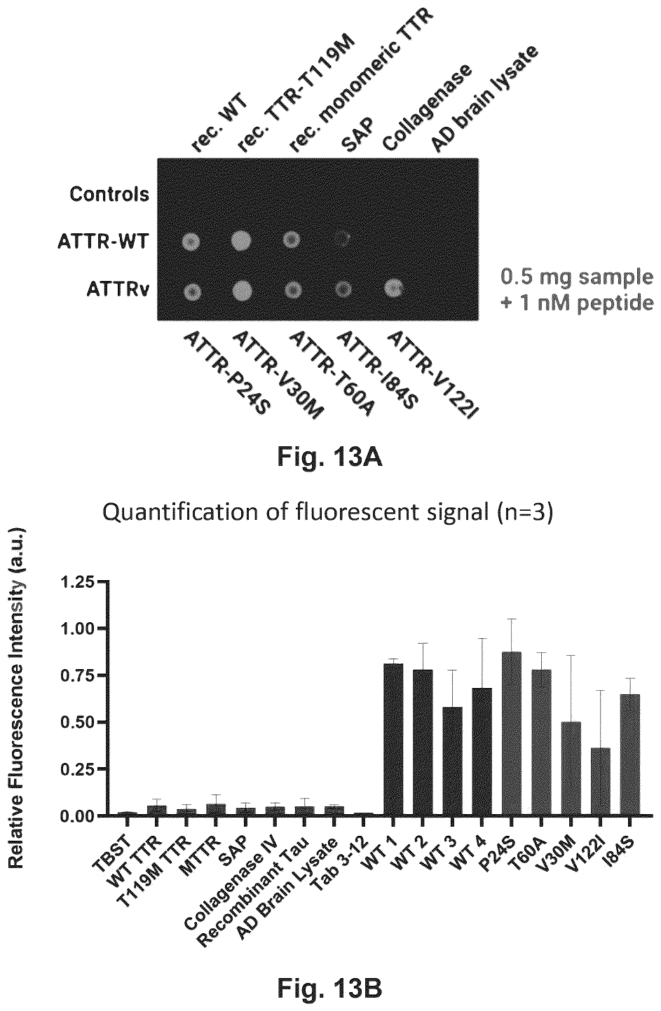

The binding of peptides to ATTR aggregate species was evaluated using dot blot analysis as we have previously described (Saelices et al., J Biol Chem. 2015 Nov. 27; 290(48):28932-43). Briefly, 0.5 μg of ex-vivo ATTR cardiac fibrils, heart lysates, recombinant protein, and control samples were dotted onto a nitrocellulose membrane (0.2 μm, Bio-Rad). The membrane was blocked for 30 minutes in 1× bovine serum albumin (BSA) in tris-buffered saline, 0.1% Tween-20 (TBST). After washing, samples were probed with TAO1 in 1×BSA/TBST for 1 hour. The fluorescence intensity of TAO1 binding was measured in an Azure Biosystems C600 imaging system through excitation of the membrane at 472 nm and reading emission at 513 nm. Fluorescence intensity of each dot was quantified through Image J and analyzed using Prism software. The results show three technical replicates. FIG. 13A and FIG. 13B show effective labeling of the probe on tissue samples, suggesting that the probe may be able to be used as a diagnostic tool in small tissue samples or biopsies.

### Example 7: Probe Affinity for Protein in Natural (Folded) State

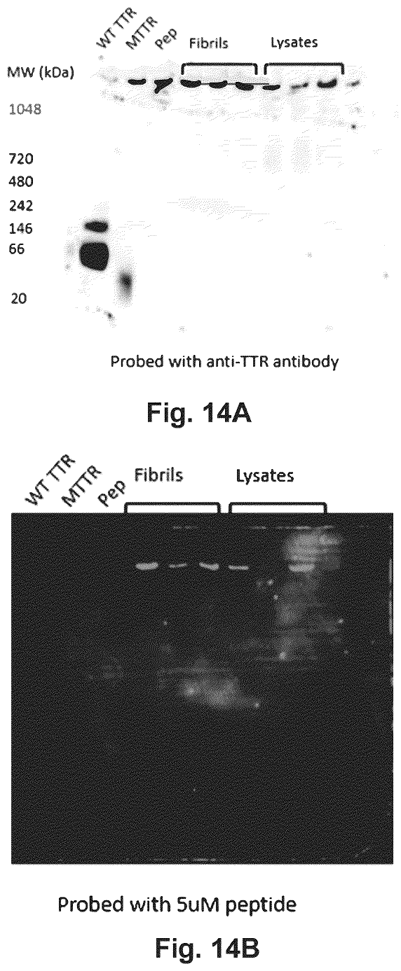

In various experiments, the affinity of the probe for folded protein, oligomers, or peptides was tested. Native gel electrophoresis was conducted using the NativePAGE Novex Bis-Tris System (Invitrogen). 5 μg of ex-vivo ATTR cardiac fibrilsJ. 5 μg of recombinant protein, or 20 μg of tissue lysate was mixed with NativePAGE 4× sample buffer according to the manufacturer's recommendation. 10 μL of each sample was loaded into a well of a NativePAGE 4-12% Bis-Tris Gel (Invitrogen). The gel was run at 150 V for approximately two hours at 4° C. and transferred onto a 0.2 μm PVDF membrane (Millipore) using the Mini Trans-Blot cell system (BioRad) for one hour at 25 V. Proteins were fixed to the membrane by incubation with 8% acetic acid. The membrane was then placed into 10% milk or 1×BSA in TBST for one hour to prevent non-specific binding. the membrane was washed three consecutive times in TBST for five minutes. The membrane was then incubated with either the anti-TTR antibody (1:1,000; Invitrogen) in 5% milk overnight, or with 5 μM peptide in 1×BSA/TBST for one hour. The membrane was then washed three times for ten minutes. The fluorescence intensity of peptide binding was measured as stated previously. The membrane probed with an anti-TTR antibody was further incubated with goat anti-rabbit secondary antibody (1:1,000; Invitrogen). The membrane was washed three times for ten minutes, and then incubated with enhanced chemiluminescence reagent (Promega). The blot was imaged in an Azure Biosystems C600 imaging system. FIG. 14A and FIG. 14B show that the probe successfully bound high molecular weight (>10 kDa) folded species, but not small oligomers, tetramers, or monomers (all in a folded state).

### Example 8: Probe Affinity for Protein in Denatured (Unfolded) State

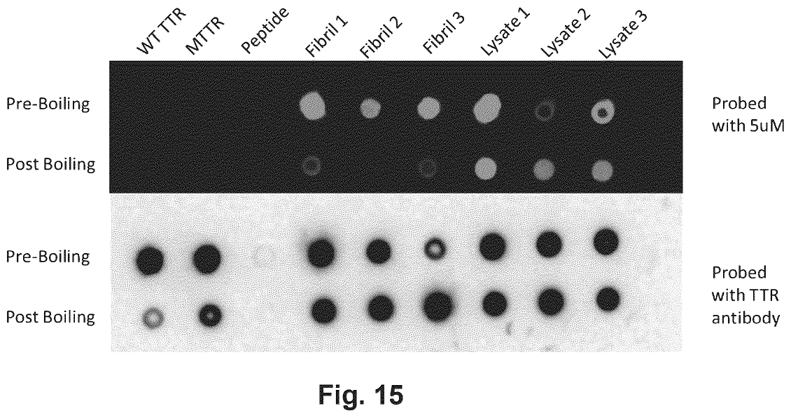

The same experiment as in Example 7 was repeated with the following changes. 5 μg of each sample (fibrils, lysates, or recombinant protein) was dotted onto a nitrocellulose membrane (0.2 μm, Bio-Rad). The same samples were also added to beta-mercaptoethanol (a denaturing agent) and boiled at 95° C. for ten minutes. These samples were dotted onto the second row of the membrane. The membrane was subjected to the same conditions as stated for the native gel experiment. It was probed with either 5 μM peptide (in which case blocking and probing step was done in BSA/1×TBST) or an anti-transthyretin antibody at a concentration of 1:1,000 (in which case blocking and probing step was completed in milk). The blots were imaged in an Azure Biosystems C600 imaging system FIG. 15 shows that the probe successfully bound high molecular weight (>10 kDa) species, but not small oligomers, tetramers, or monomers (in an unfolded state). Together, data shown in Examples 7 and 8 (FIGS. 14A, 14B, and 15) show that the probe preferentially binds high molecular weight species, but not small oligomers, tetramers or monomers in either a folded or unfolded state.

### Example 9: Probe Validation in Patient Samples

The ability of the peptide probes to bind ATTR fibrils in different patient samples will be tested. Plasma, serum, and tissue biopsy samples will be obtained from patients with symptomatic ATTRwt (with cardiac phenotype), symptomatic ATTRv (with cardiac phenotype), symptomatic ATTRv (without cardiac phenotype), symptomatic AL (light chain amyloidosis characterized by aggregation of immunoglobulin light chain amyloid protein), symptomatic AA (another amyloidosis characterized by aggregation of serum amyloid A) or patients with any other systemic amyloidosis as well as control asymptomatic non-carriers. To the extent possible, the patient cohort will be age and sex matched. Samples will be treated and processed as described previously and labeled with a peptide probe of the present disclosure.

### Example 10: Monitoring Treatment Outcomes

Treatment outcomes will be monitored using a polypeptide probe of the present disclosure. Specifically, tissue samples (plasma, serum and tissue biopsy) will be obtained before and after treatment (with stabilizers and gene silencers) from patients with symptomatic ATTRv (with cardiac phenotype), symptomatic ATTRv (without cardiac phenotype), and ATTRwt (with cardiac phenotype) as well as control non-carriers. Treatment outcomes will be correlated with detected levels of ATTR in each patient sample.

### Example 11: Early Detection with Polypeptide Probes

A further experiment will be conducted to measure levels of ATTR in plasma and tissue biopsy samples obtained from pre-symptomatic ATTRwt patients (with cardiac phenotype), pre-symptomatic ATTRv patients (with cardiac phenotype), pre-symptomatic ATTRv patients (without cardiac phenotype), symptomatic ATTRwt patients (with cardiac phenotype), symptomatic ATTRv patients (with cardiac phenotype), symptomatic ATTRv patients (without cardiac phenotype), and in patients considered to be at risk of developing ATTR amyloidosis, such as carpal tunnel patients, lumbar spinal stenosis patients, and patients with other orthopedic manifestations. This group may also encompass lifetime older high intensity athletes and patients with heart failure with preserved ejection fraction (HFpEF). Levels of ATTR in samples in pre-symptomatic patients will be correlated with symptom presentation and other markers of disease severity.

### Example 12: Materials and Methods for Examples 13-19

Peptide design and synthesis. Peptide development was performed by rational design starting from peptide inhibitors that target the two amyloid-driving segments of TTR (described in PCT/US17/40103, which is incorporated herein by reference in its entirety). All sequences are listed in Table 1, discussed below. Fluorescent and epitope modifications were added to peptides to allow for detection. Peptides were synthesized by LifeTein LLC. and sent to use in lyophilized form. Prior to running experiments, the peptide was reconstituted in distilled water to yield a final concentration of 1 mM. The resuspended peptide was filtered through a 0.22 μm filter tube by spinning at 20,000×g for 5 minutes at 4° C. to remove any possible aggregates from the solution.

Patients and tissue material. Cardiac tissues from ATTR patients carrying wild-type TTR (n=4) or TTR mutations (n=7) included in the study are listed in Supplementary Table 2. Specimens from the left ventricle of either explanted or autopsied hearts were obtained from the laboratory of the late Dr. Merrill D. Benson at the University of Indiana. Serum and plasma samples of ATTR patients carrying wild-type TTR (n=88) or TTR mutations (n=34), as well as healthy controls (n=32) and ATTRv carriers without cardiac symptoms (n=16) are listed in Supplementary Table 3. Serum and plasma specimens were obtained from Dr. Ahmad Masri at Oregon Health and Science University, Ors. Wilson Tang and Mazen Hanna from Cleveland Clinic, and the Dallas Heart Study at UTSW. The Office of the Human Research Protection Program granted exemption from Internal Review Board review because all specimens were anonymized.

Extraction of amyloid fibrils from human cardiac tissue. Ex vivo fibrils were extracted from fresh-frozen heart tissue as described by Nguyen, Afrin, et al bioRxiv, 2022.2006.2021.496949 (2022), which is incorporated herein by reference in its entirety. Briefly, ˜100 mg of frozen cardiac tissue per patient was thawed at room temperature and cut into small pieces with a scalpel. The minced tissue was suspended in 400 μL Tris-calcium buffer (20 mM Tris, 138 mM NaCl, 2 mM CaCl₂, 0.1% NaN₃, pH 8.0) and centrifuged for 5 minutes at 3, 100×g and 4° C. The pellet was washed and centrifuged in Tris-calcium buffer three additional times. After washing, the pellet was resuspended in 375 μL of 5 mg/ml collagenase (Sigma Aldrich) in Tris-calcium buffer. This solution was incubated overnight at 37° C., shaking at 400 rpm. The resuspension was centrifuged for 30 minutes at 3100×g and 4° C. and the pellet was resuspended in 400 μL Tris-ethylenediaminetetraacetic acid (EDTA) buffer (20 mM Tris, 140 Mm NaCl, 10 mM EDTA, 0.1% NaN₃, pH 8.0). The suspension was centrifuged for 5 minutes at 3,100×g and 4° C., and the washing step with Tris-EDTA was repeated nine additional times. After the washing, the pellet was resuspended in 200 μLice-cold water supplemented with 5 mM EDTA and centrifuged for 5 minutes at 3, 100×g and 4° C. This step released the amyloid fibrils from the pellet, which were collected in the supernatant. EDTA helped solubilize the fibrils. This extraction step was repeated five additional times. The material from the various patients was handled and analyzed separately.

Preparation of fibril seeds. Extracted fibrils were treated with 1% sodium dodecyl sulfate, and the soluble fraction was discarded after centrifugation at 13,000 rpm for 5 minutes. This process was repeated twice, and the supernatants were discarded each time. The sample was washed with 10 mM sodium acetate (pH 7.5), 100 mM KCl, and 1 mM EDTA three times then sonicated in cycles of 5 son/5 s off for a total of 10 min. The protein content was measured using the Pierce Micro BCA Protein Assay Kit (Thermo Fisher Scientific).

Thioflavin screening of peptides. A thioflavin T (ThT) fluorescence assay was used as an indirect measure of peptide binding to fibrils according to published protocols (Saelices, L. et al Proc Natl Acad Sci USA 115, E6741-E6750 (2018) and Saelices, L. et al. J Biol Chem 294, 6130-6141 (2019)). The binding of peptides to fibril seeds delays or inhibit seeding of soluble TTR and the formation of amyloid fibrils, which is monitored by the fluorescence of ThT. 30 ng/ml seeds were added to 0.5 mg/ml recombinant transthyretin at a final volume of 200 μL of 5 μM ThT, 10 mM sodium acetate (pH 7.5), 100 mM KCl, and 1 mM EDTA. Plates were incubated at 37° C. for 132 hours shaking at 700 rpm. ThT fluorescence emission was measured at 482 nm with absorption at 440 nm in a FLUOstar Omega (BMG LabTech) microplate reader.

Preparation of patient-derived crude heart lysates. For preparation of crude tissue lysates, 5 mg of patient heart tissue was suspended in 500 μL Tris-calcium buffer containing a protease inhibitor cocktail (Sigma Aldrich). The sample was homogenized using a biomasher (Polysciences) for 5 minutes. The sample was then placed into a bath sonicator where it was pulsed 5 seconds on, 5 seconds off for ten minutes at amplitude 80. The protein concentration within the lysate was determined using the Pierce Micro BCA protein assay kit (Thermo Scientific).

Fluorescent immunodot blotting of extracted fibrils, crude heart lysates, and blood samples. The binding of TAD1 to ATTR species was evaluated using immunodot blot analysis as we have previously described (Saelices, L. et al. J Biol Chem 290, 28932-28943 (2015)). Unless otherwise stated, 0.5 μg of ex-vivo ATTR cardiac fibrils, heart lysates, recombinant protein, and control samples were dotted onto a nitrocellulose membrane (0.2 μm, Bio-Rad). For assays using blood samples, 30 μL of sample was dotted onto the membrane unless otherwise noted. The membrane was blocked for 30 minutes in 1× bovine serum albumin (BSA) in tris-buffered saline, 0.1% Tween-20 (TBST). After washing, samples were probed with TAD1 in 1×BSA/TBST for 1 hour or overnight for blood samples. The fluorescence intensity of TAD1 binding was measured in an Azure Biosystems C600 imaging system through excitation of the membrane at 472 nm and reading emission at 513 nm. TAD1 fluorescence intensity was quantified using the ImageJ software. Signal was normalized to the highest fluorescence intensity on the membrane. For blood samples, the signal was normalized with respect to the intensity of ATTRwt fibrils.

In vitro aggregation assay of TTR. Generation of recombinant transthyretin aggregates was done as described previously (Saelices, L. et al. J Biol Chem 290, 28932-28943 (2015)). Briefly, 1 mg/ml of tetrameric transthyretin with an N-terminal polyhistidine tag was incubated in 10 mM sodium acetate (pH 4.3), 100 mM KCl, and 1 mM EDTA at 37° C. for four days. At the specified time points, 35 μL of the reaction was removed at each time point for anti-TTR, and TAD1 immunodot blotting as described above. Additionally, 50 μL of the reaction was collected and spun at 13,000 rpm for 30 minutes to pellet insoluble aggregates. The supernatant was removed, the pellet was resuspended in 50 μL fresh aggregation buffer, and the centrifugation step was repeated. After the second centrifugation, the supernatant was removed, and the pellet was resuspended in 50 μL 6 M guanidine hydrochloride. 2 μL of this mixture was added to 18 μL of guanidine hydrochloride before analysis. The polyhistidine tag in the insoluble fraction was probed with the SuperSignal West HisProbe Kit (ThermoFisher Scientific) according to the manufacturer's recommendation with the following modification: instead of probing with 1:5,000 HisProbe-HRP working solution, the membrane was probed with 1:20,000 working solution.

Denaturing western blot of extracted fibrils and crude heart lysates. 1 μg of recombinant protein or 5 μg of ex-vivo ATTR cardiac fibrils were boiled at 95° C. for ten minutes. The samples were loaded onto three independent SurePAGE Bis-Tris 1O×84-12% gel (Genscript) was run at 150 V for approximately one hour. One gel was treated for immunostaining using and anti-TTR polyclonal antibody, one gel was treated for the detection of fluorescence upon binding to TAD1, and one gel was used as a control for staining with Coomassie. Two gels were transferred onto nitrocellulose membranes at 25 V for 16 minutes using the Trans Blot Turbo System (BioRad). The membranes were placed into 10% milk or 1×BSA in TBST for one hour to prevent non-specific binding and washed three consecutive times in TBST for five minutes. One membrane incubated with the anti-TTR antibody (1:1,000; Genscript) in 5% milk overnight, and the other membrane was incubated with 5 μM TAD1 in 1×BSA/TBST for one hour. The membranes were then washed three times for ten minutes. The fluorescence intensity of TAD1 binding was measured as stated above. The membrane probed with an anti-TTR antibody was further incubated with goat anti-rabbit secondary antibody (1:1,000; Invitrogen). The membrane was washed three times for ten minutes, and then incubated with enhanced chemiluminescence reagent (Promega). The blot was imaged in an Azure Biosystems C600 imaging system. The third gel was stained with coomassie using the SimplyBlue SafeStain (Thermo Fisher Scientific). The gel was washed with distilled water three times for five minutes each to remove sodium dodecyl sulfate. It was then microwaved in distilled water for one minute and thirty seconds, followed by shaking for one minute. This process was repeated two more times. The distilled water was then exchanged for SimplyBlue SafeStain and microwaved for thirty seconds. The gel was incubated in dye for 10 minutes, then destained in distilled water and imaged in the Azure Biosystems C600.

Native gel electrophoresis of extracted fibrils and crude lysates. Native gel electrophoresis was conducted using the NativePAGE Novex Bis-Tris System (Invitrogen). 5 μg of ex-vivo ATTR cardiac fibrils, 5 μg of recombinant protein, or 20 μg of tissue lysate was mixed with NativePAGE 4× sample buffer according to the manufacturer's recommendation. 10 μL of each sample was loaded into a well of three independent NativePAGE 4-12% Bis-Tris Gels (Invitrogen). The gels were run at 150 V for approximately two hours at 4° C. and transferred onto 0.2 μm PVDF membranes (Millipore) using the Mini Trans-Blot cell system (BioRad) for one hour at 25 V. Proteins were fixed to the membrane by incubation with 8% acetic acid for 15 minutes. The membranes were then subjected to the same staining protocol as described earlier, with the following modifications: the membranes were incubated overnight with either a primary anti-TTR antibody (1:1,000; Genscript) or 5 μM TAD1. For Coomassie staining, the gel was run using the dark blue cathode buffer as recommended by the manufacturer. After running the gel, the gel was placed in a fixing solution (40% methanol, 8% acetic acid), microwaved for 45 seconds and placed on an orbital shaker for 15 minutes. The solution was then decanted, and the gel was placed in distain solution (8% acetic acid) until the desired background was obtained.

Filtration assay with patient plasma. 60 μL of plasma samples from ATTR patients and healthy controls were subjected to filtration using a 0.22 μM centrifugal filter tube (Corning). The samples were spun at 10 second intervals at 1000 g, 4° C. until there was 20 μL of filtrate at the bottom of the tube. 20 μL of unfiltered plasma, plasma that did not pass through the filter (void) and plasma that did pass through the 0.22 μM filter (filtrate) were dotted onto nitrocellulose membrane and probed with 10 μM TAD1 and anti-TTR antibody as described above.

Native Gel Shift Assay with TAD1. 30 μg of ATTRwt patient plasma or healthy control plasma was incubated overnight with increasing concentrations of TAD1 (0, 12.5, 25, 50, 100 μM TAD1). The samples were then subjected to gel electrophoresis under non-denaturing conditions followed by western blotting with an anti-TTR antibody as described above. For quantification, the banding patterns were segmented into three groups (high molecular weight aggregates, oligomers, and tetramers) based on their associated molecular weight. These bands were quantified using ImageJ software (Schneider, C. A., Rasband, W. S. & Eliceiri, K. W. NIH Nat Methods 9, 671-675 (2012)).

Immunogold labeling of ex vivo fibrils. 5 μM TAD1 was mixed with 50 nM or 250 nM Ni-NTA Nanogold (Nanoprobes) in binding buffer (20 mM Tris pH 7.6, 150 mM NaCl) overnight at 4° C. The next day the nanogold beads pelleted to the bottom of the reaction mixture. The supernatant containing unbound nanogold beads was removed. 1 μg/ml of fibrils extracted from a ATTRwt patient was spiked into the pellet and the solution was loaded onto a glow-discharged carbon coated electron microscopy grid (Electron Microscopy Sciences, copper film 300 mesh). The solution was incubated for two minutes before using filter paper to remove excess liquid. The grid was stained with 1% uranyl acetate for one minute. The grid was blotted again to remove excess stain and then visualized using a Fei Tecnai Spirit transmission electron microscope (4K FEI CCD camera).

Statistical Analysis. Statistical analysis of TAD1 fluorescence, TTR aggregation, and ThT signal was conducted using the GraphPad Prism software. All samples were included in the analysis. All quantitative experiments were performed using three independent replicates and are reported as means±standard deviation of these replicates. Statistical significance between groups were compared using a one-way ANOVA, where the cutoff of p<0.05 was used to determine statistical significance between groups. Outliers in each data set were identified and removed using a Grubbs test.

### Example 13: Development of Peptide Probes

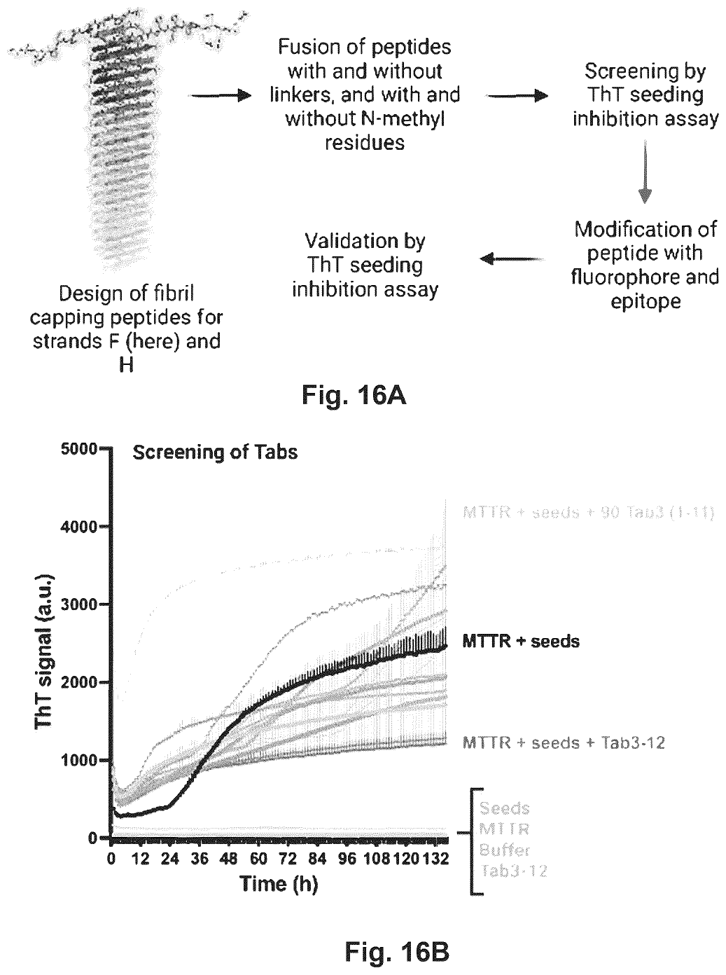

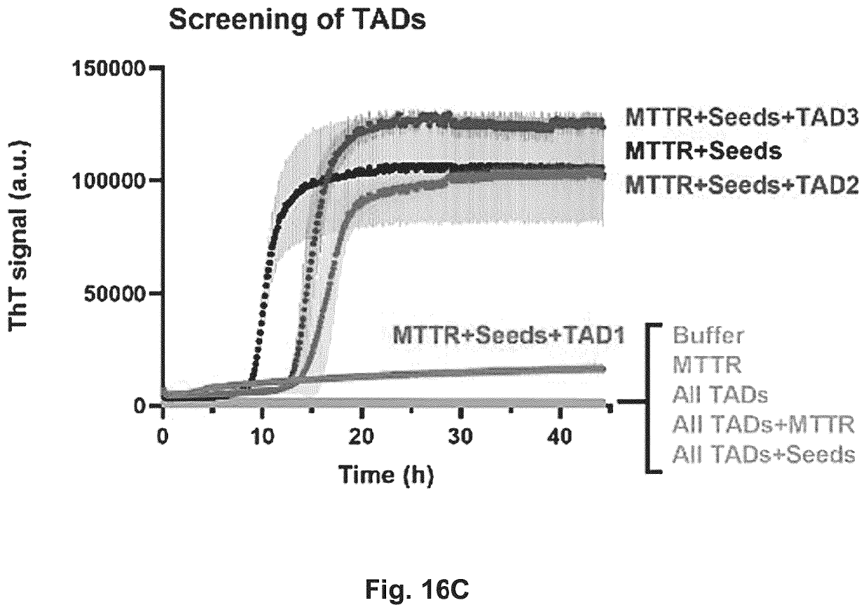

The workflow for the design of novel peptide probes is shown in FIG. 16A. In previous studies, first and second-generation transthyretin aggregation blocker peptides (TAB) independently targeting the two amyloid-driving segments of transthyretin, 13 strands F and H were developed (see e.g., Saelices, L. et al. Biol Chem 290, 28932-28943 (2015); Saelices, L. et al Proc Natl Acad Sci USA 115, E6741-E6750 (2018) and Saelices, L. et al. J Biol Chem 294, 6130-6141 (2019), which are each incorporated herein by reference in their entirety). These peptides were designed to cap the tip of the fibrils to inhibit protein aggregation and fibril elongation in vitro and in vivo. It was found that these peptides inhibited a process known as amyloid seeding, in which mature ex-vivo ATTR fibrils catalyze the formation of de novo amyloid fibrils by the templated addition of soluble recombinant transthyretin. In the present study, the specific binding of these TAB peptide inhibitors was leveraged to repurpose them into detection probes. First, these peptides were optimized to generate third-generation TAB peptide inhibitors (TAB3) (Table 1).

TABLE 1
Third generation peptide inhibitor sequences.

| Pep- | | SEQ |
|---|---|---|
| tide | | ID |
| name | Sequence | NO: |
| Tab3- | RRRRHVAHPFVEFTE | 13 |
| 01 | | |
| Tab3- | RRRRSYVTNPTSYAVT | 21 |
| 02 | | |
| Tab3- | RRRRHVAHPFVEFTERRRRSYVTNPTSYAVT | 32 |
| 03 | | |
| Tab3- | RRRRSYVTNPTSYAVTRRRRHVAHPFVEFTE | 33 |
| 04 | | |
| Tab3- | RRRRHVAHPFV*EFTERRRRSYVTNPTSY*A | 34 |
| 05 | VT | |
| Tab3- | RRRRSYVTNPTSY*AVTRRRRHVAHPFV*EF | 35 |
| 06 | TE | |
| Tab3- | RRRRHVAHPFVEFTEGSRRRRSYVTNPTSYA | 36 |
| 07 | VT | |
| Tab3- | RRRRHVAHPFVEFTEGGSRRRRSYVTNPTSY | 37 |
| 08 | AVT | |
| Tab3- | RRRRHVAHPFVEFTEGGGSRRRRSYVTNPTS | 38 |
| 09 | YAVT | |
| Tab3- | RRRRHVAHPFVEFTEGGGSTRRRRSYVTNPT | 39 |
| 10 | SYAVT | |
| Tab3- | RRRRHVAHPFVEFTEGGGSARRRRSYVTNPT | 40 |
| 11 | SYAVT | |
| Tab3- | RRRRHVAHPFVEFTEGGGSTERRRRSYVTNP | 9 |
| 12 | TSYAVT | |
| Tab3- | RRRRHVAHPFVEFTEGGGSAERRRRSYVTNP | 41 |
| 13 | TSYAVT | |

*N-methyl modification on the preceding residue

To screen and validate the TABs, their inhibitory effect of amyloid seeding was assessed using thioflavin T (ThT) assays and ex-vivo ATTRwt fibril seeds, prepared previously described (see e.g., Saelices, L. et al Proc Natl Acad Sci USA 115, E6741-E6750 (2018) and Saelices, L. et al. J Biol Chem 294, 6130-6141 (2019)). As it was found that TAB3-12 (SEQ ID NO: 9) was the most potent inhibitor (FIG. 168, blue), this peptide was selected for further modification. TAB3-12 was fused to an N-terminal epitope and a fluorescent tag, to generate first-generation transthyretin aggregation detectors (TADs). The binding of TADs to fibrils was screened using an amyloid seeding inhibition assay as a proxy, as performed for peptide inhibitors, and found that TAD1 (containing a polyhistidine tag) did not induce fibril formation, and fully inhibited seeding instead (FIG. 16C). The sequences of all three TAD detection probes (TAD1, TAD2 and TAD3) are provided in Table 2, below. In the table, TAB3-12 (RRRRHVAHPFVEFTEGGGSTERRRRSYVTNPTSYAVT, SEQ ID NO: 9) is underlined in each probe.

| Pep- | Sequence | | |
|---|---|---|---|
| tide | (SEQ ID | N-Terminal | N-Terminal |
| name | NO: ) | Modification | Epitope Tag |
| TAD1 | HHHHHHRRRRHVA | FITC- | Polyhistidine |
| | HPFVEFTEGGGST | Aminohexanoic | |
| | ERRRRSYVTNPTS | acid (Ahx) | |
| | YAVT | | |
| | (SEQ ID NO: | | |
| | 12) | | |
| TAD2 | YPYDVPDYARRRR | FITC-Ahx | Hemagglutinin |
| | HVAHPFVEFTEGG | | |
| | GSTERRRRSYVTN | | |
| | PTSYAVT | | |
| | (SEQ ID NO: | | |
| | 10) | | |
| TAD3 | DYKDDDDKRRRRH | FITC-Ahx | FLAG |
| | VAHPFVEFTEGGG | | |
| | STERRRRSYVTNP | | |
| | TSYAVT | | |
| | (SEQ ID NO: | | |
| | 11) | | |

It was also confirmed that TAD1 did not form fibrils by itself and TAD2 and TAD3 did not inhibit amyloid seeding fully (FIG. 16C). Therefore, TAD1 (SEQ ID NO: 12) was selected for further studies.

### Example 14: Dot Blotting as a Simple Experimental Platform for Analysis of Patient Samples

As described in earlier examples (e.g., Examples 2-10), dot blotting was chosen as an experimental platform to measure direct binding of TAD1 to different samples (FIG. 17) because it is technically and conceptually simple, does not discriminate sample types, and provides an unquestionable readout of direct binding between two molecules. In brief, the study samples were loaded onto a nitrocellulose membrane before incubating the membrane with TAD1, washing the membrane to remove excess probe and visualization of TAD1 binding to the study sample using fluorescence. A greater dot intensity indicates more TAD1 binding to the sample. The relative intensity can then be quantified to show relative TAD1 binding.

### Example 15: TAD1 Binds Patient Derived ATTR Fibrils and ATTR within Tissue Lysates

Further to the experiments and data described in Examples 2-10 and especially in Example 3, the ability of TAD1 to bind ATTR fibrils purified from ATTR amyloidosis patients and present in ATTR heart lysates was tested. Fibrils were extracted from fresh frozen cardiac tissue from nine patients, four wild types (ATTRwt) and five hereditary variants (ATTRv), as performed before using the method described by Nguyen, Afrin et al bioRxiv, 2022.2006.2021.496949 (2022), and in paragraph [0131], above. Table 3 below provides a list of all ATTR cardiac tissue samples included in the study.

TABLE 3
List of ATTR cardiac tissue samples included in the study

| Genotype | Origin | Sex | Age at collection | Neuropathy signs |
|---|---|---|---|---|
| ATTRwt-1 | Autopsy | Male | 84 | No |
| ATTRwt-2 | Autopsy | Male | 83 | No, butamyloid |
| | | | | found in autopsy |
| ATTRwt-3 | Autopsy | Male | 70 | No, butamyloid |
| | | | | found in autopsy |
| ATTRwt-4 | Transplant | Male | 78 | No |
| ATTRv P24S | Transplant | Male | 65 | No |
| ATTRv V30M | N/A | N/A | N/A | N/A |
| ATTRv T60A | Autopsy | Male | 57 | Moderate |
| ATTRv I84S | Autopsy | Male | 53 | Mild |
| ATTRv V122I | Autopsy | Male | Unknown | Unknown |

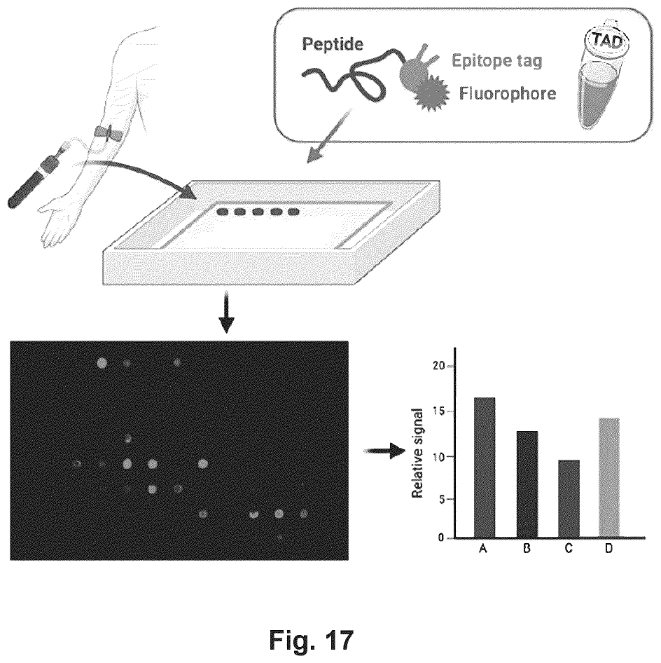

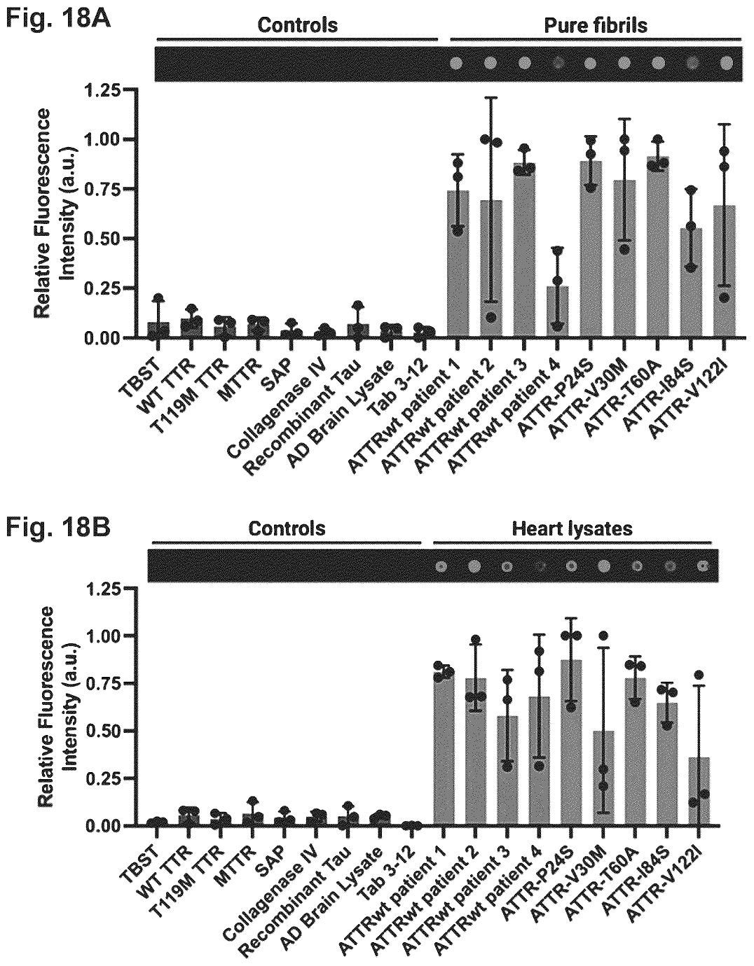

5 μg of fibrils were loaded along with controls on the nitrocellulose membrane that was incubated with TAD1. The controls included recombinant tetrameric TTR, recombinant TTR with a T119M mutation, recombinant monomeric TTR variant (MTTR), TAB3-12, serum amyloid protein (SAP, which can associate with ATTR fibrils), and collagenase (used in the fibril extraction process). To test the specificity of TAD1 for ATTR fibrils and not just proteins in a fibrillar conformation, recombinant tau fibrils and brain lysate from an Alzheimer's Disease (AD) patient were used as controls. It was found that TAD1 can be used for the detection of purified ATTR fibrils regardless of genotype of the ATTR patient (FIG. 18A). Similarly, 5 μg of heart lysates from the same patients as in FIG. 18A were loaded along with the same controls onto a membrane that was incubated with 5 μM TAD1 (FIG. 18B). With this technique, ATTR species present in crude tissue lysates obtained from these patients were detected (FIG. 18B). There is no detectable TAD1 binding to control samples in either experiment (FIGS. 18A-18B). These experiments showed that TAD1 can be used for the detection of patient derived ATTR fibrils and ATTR fibrils within lysates in a specific manner, thereby validating the probe design strategy.

### Example 16: TAD1 Binds ATTR Fibrils with High Sensitivity and Precision

The sensitivity and precision of the TAD1 dot blotting system was then evaluated. To determine TAD1 sensitivity, different amounts of ATTRwt fibrils were titrated on a membrane that was incubated with 5 M TAD1. It was found that TAD1 displays high sensitivity, recognizing approximately 20 ng of purified ATTRwt fibrils (FIG. 19A) with a calculated EC50 of 26.1 ng (FIG. 19B) (see also, FIG. 4, Example 3). To assess the precision of the experimental setup and variability of the fluorescence readouts, three different amounts of ATTRwt fibrils (1.5 ng, 3 ng and 6 ng) were loaded onto a membrane and probed with 5 μM TAD1 (FIG. 19C). The quantification of the fluorescence signal shows that TAD1 can distinguish between small variations in the amount of fibrils with statistical significance (FIG. 19D). These results indicate that the probe and experimental setup display high sensitivity and precision and further support data presented in Example 3 above.

### Example 17: TAD1 Reveals a Novel Blood Biomarker in Cardiac ATTR Amyloidosis Patients

Transthyretin can adopt non-native conformations in blood of neuropathic ATTRv amyloidosis patients that could represent early stages of protein aggregation. Since TAD1 displays high sensitivity for ATTR fibrils and not for other forms of transthyretin, it was hypothesized that TAD1 could detect the presence of these non-native ATTR species (or different ones) in blood. Thus, it was tested whether serum and plasma samples from both ATTRwt and ATTRv amyloidosis patients (Table 4) contain TAD1-positive species.

TABLE 4
Characteristics of cardiac ATTR patient, carrier, AL patient,
and control blood included in the study

| | | Pre- | Post- | | AL |
|---|---|---|---|---|---|
| | | Treatment | Treatment | ATTRv | Amyloidosis |
| Demographic | Controls | Patients | Patients | Carriers | Patients |
| Characteristics | (n = 33) | (n = 39) | (n = 43) | (n = 16) | (n = 10) |
| Age in years, | 50.94 | 77.60 | 75.49 | 52.07 | 71.63 |
| mean (SD) | (10.5) | (6.8) | (7.7) | (12.4) | (9.56) |
| Male, n (%) | 11 | 35 | 37 | 2 | 5 |
| | (33.3) | (89.7) | (86.1) | (13.3) | (50.0) |
| Clinical | | | | | |
| Characteristics | | | | | |
| Type of ATTR | | | | | |
| WT, n (%) | N/A | 32 | 31 | 0 | N/A |
| | | (82.1) | (72.1) | | |
| Variant, n (%) | N/A | 7 | 12 | 16 | N/A |
| | | (17.9) | (27.9) | (100) | |
| Variant Types | | | | | |
| ATTR-V30M | N/A | 5 | 4 | 6 | N/A |
| | | (12.8) | (33.3) | (40.0) | |
| ATTR-V32A | N/A | 0 | 0 | 1 | N/A |
| | | | | (6.7) | |
| ATTR-T60A | N/A | 0 | 6 | 2 | N/A |
| | | | (50.0) | (13.3) | |
| ATTR-S97Y | N/A | 1 | 0 | 0 | N/A |
| | | (2.6) | | | |
| ATTR-V1221 | N/A | 0 | 2 | 5 | N/A |
| | | | (16.7) | (33.3) | |
| Unknown | N/A | 1 | 0 | 1 | N/A |
| | | (2.6) | | (6.7) | |
| Treatment Type | | | | | |

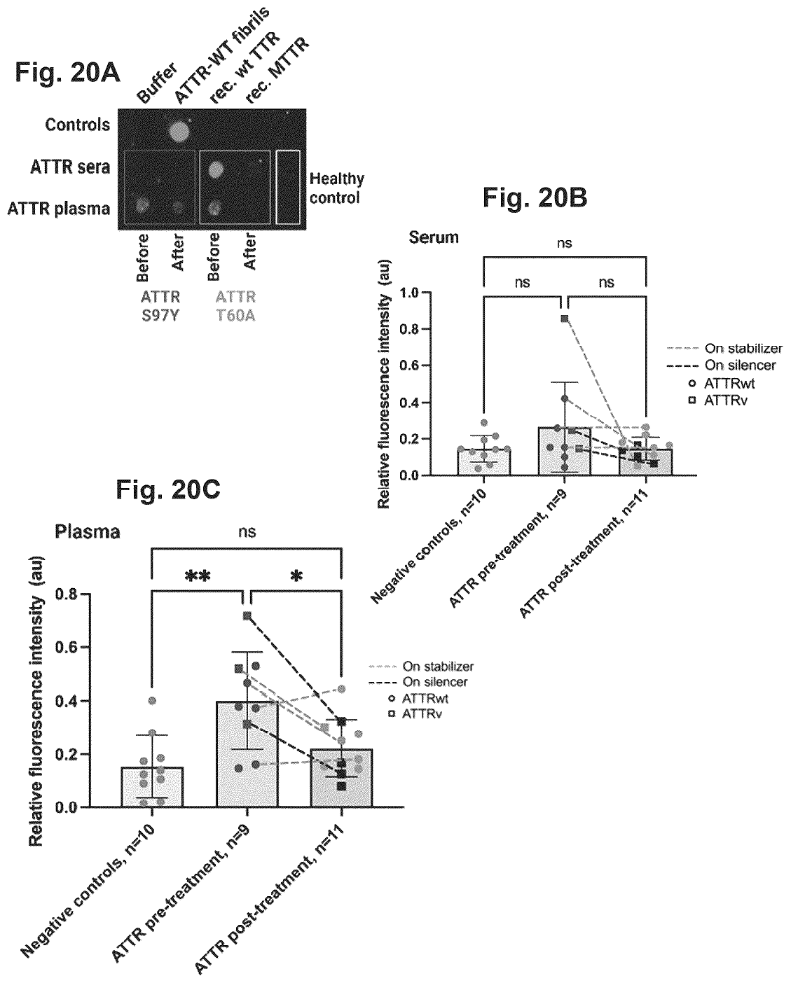

First, a pilot dot blotting assay of two ATTR samples and controls was run by probing with 5 μM TAD1. It was found that both serum and plasma contain ATTR species that are detected by TAD1 (FIG. 20A). Next, a small cohort of patient samples was analyzed to investigate which sample type (serum or plasma) could provide better readouts of the presence of these species (FIG. 208). In serum, quantifying relative fluorescence intensity of TAD1 reveals no distinct difference in binding when comparing treated patients, untreated patients, and negative controls (FIG. 208). In contrast, the analysis of plasma samples revealed a significant difference between negative controls and ATTR patients pre-treatment, including both ATTRwt and ATTRv patients (FIG. 20C). It was also found that there was a significant difference in TAD1 signal between pre-treatment and post-treatment regardless of the treatment type (FIG. 20C). These experiments indicate that serum does not contain TAD1-positive species detectable under these conditions, and so further studies were performed using plasma samples.

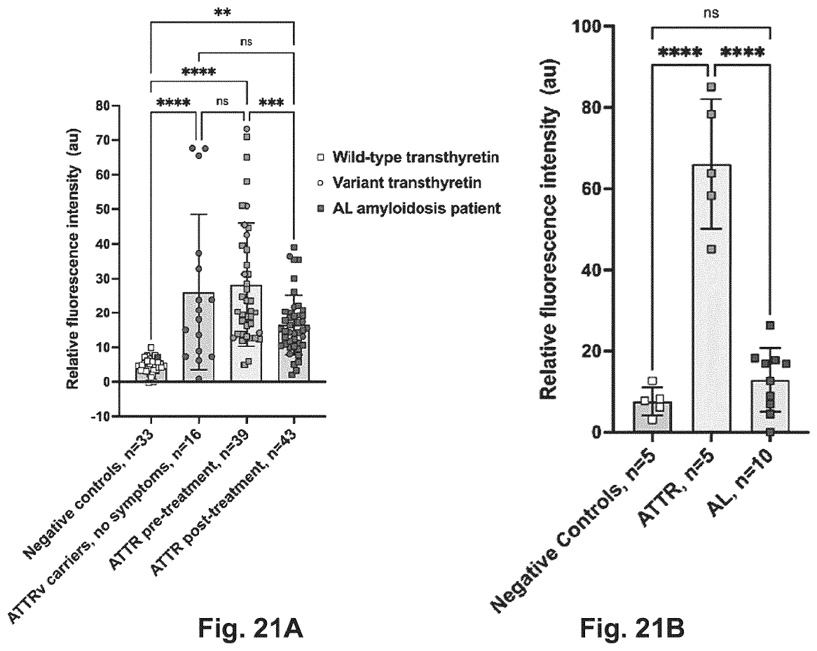

The pilot study data described above was then expanded to test TAD1 binding to species in plasma of a larger cohort of samples, including healthy sex- and age-matching controls, ATTR amyloidosis patients pre- and post-treatment, ATTRv carriers prior to showing symptoms, and immunoglobulin light chain amyloidosis (AL amyloidosis) patients (see Table 4, above). It was found that TAD1 signal is significantly higher in ATTR patients (pre-treatment) when compared to healthy controls (FIG. 21A). Significant differences in TAD1 signal between ATTR patients pre- and post-treatment were also observed (FIG. 21A). Additionally, TAD1 signal in ATTRv carriers was detected prior to showing cardiac manifestations, indicating that TAD1 may be used as a tool for early detection of these unique ATTR species in plasma (FIG. 21A). There is no detectable TAD1 signal in plasma from AL amyloidosis patients, further showing the specificity of TAD1 for ATTR (FIG. 218). These results validate the earlier data presented in Examples 2-10 and suggest that ATTR plasma contains a unique biomarker that decreases in response to treatment and appear in blood prior to symptoms. This biomarker may represent a powerful tool for early detection and monitoring treatment response.

### Example 18: TAD1 Binds Large Species from Recombinant Protein, ATTR Fibril Extracts, And Plasma

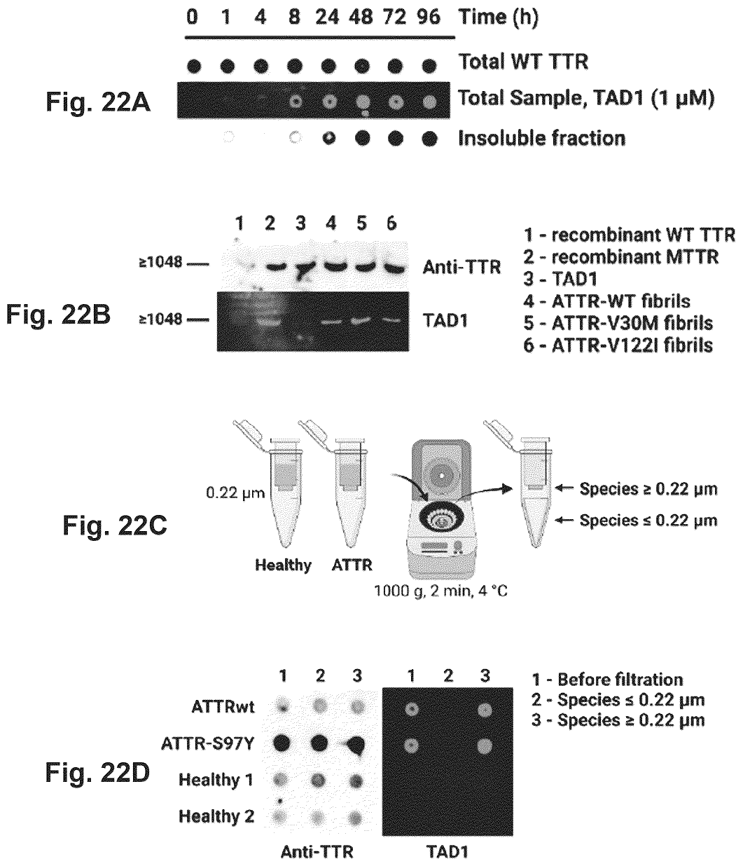

It was unclear what the molecular nature of the detected ATTR species is in the patient samples. It was hypothesized that since TAD1 binds ATTR fibrils with a high affinity, the detected species in patient samples may be high molecular weight ATTR aggregates. This hypothesis was tested using three types of samples: recombinant TTR aggregates, purified ex-vivo ATTR fibrils, and plasma samples from ATTR patients (FIGS. 22A-22D and FIGS. 24A-248).

First, the binding of TAD1 to recombinant TTR aggregates made under acidic pH using wild-type transthyretin was evaluated as described previously (see e.g., Saelices, L. et al. J Biol Chem 290, 28932-28943 (2015)). In this standard procedure, the native tetrameric structure of transthyretin is dissociated to promote aggregation in vitro by lowering the pH of the sample to 4.3 and monitor aggregation by measuring absorbance at 400 nm. After performing this assay, aggregation can be visualized by dot blotting using an antibody that recognizes the polyhistidine tag of recombinant transthyretin (FIG. 22A). Whereas the total amount of transthyretin does not seem to change in the sample (FIG. 22A, upper panel), TAD1-positive species increase overtime (FIG. 22A, middle panel). TAD1 signal correlates with an increase in insoluble aggregates collected by centrifugation (FIG. 22A, bottom panel), suggesting that TAD1 binds recombinant aggregates in a conformation-dependent manner.

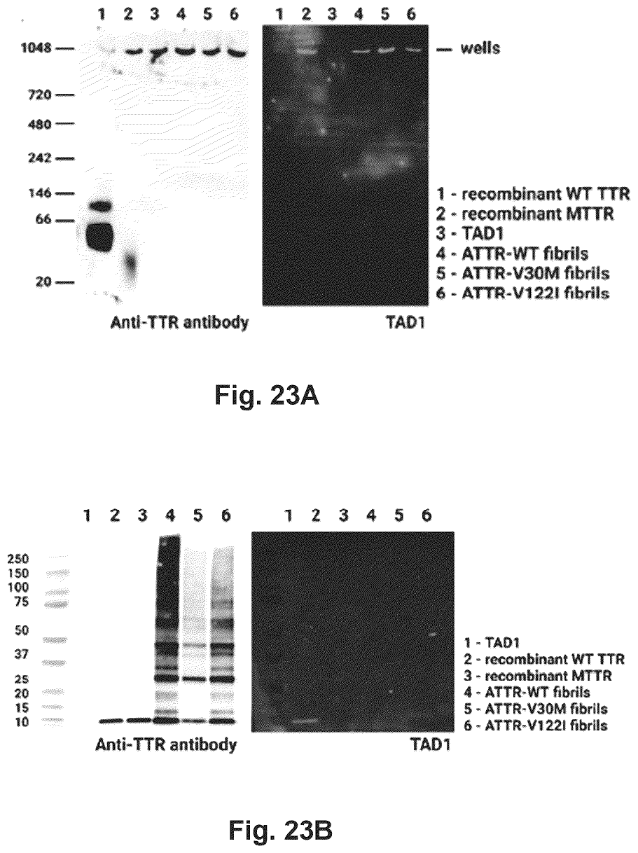

To characterize the size of TAD1-positive species in purified fibrils, western blotting was performed under non-denaturing and denaturing conditions (FIG. 22B and FIG. 23A and FIG. 238). It was observed that in non-denaturing conditions, TAD1 binds to ATTR fibrils and recombinant transthyretin aggregates that do not run through the gel. These aggregates correspond to a molecular weight higher than 1048 kDa (FIG. 228, FIG. 23A). When the same samples are subjected to denaturing conditions, TAD1 loses its ability to bind aggregates or fragments that result from denaturing ATTR fibrils (FIG. 23B), providing additional evidence that the recognition of TAD1 to ATTR species is conformational.

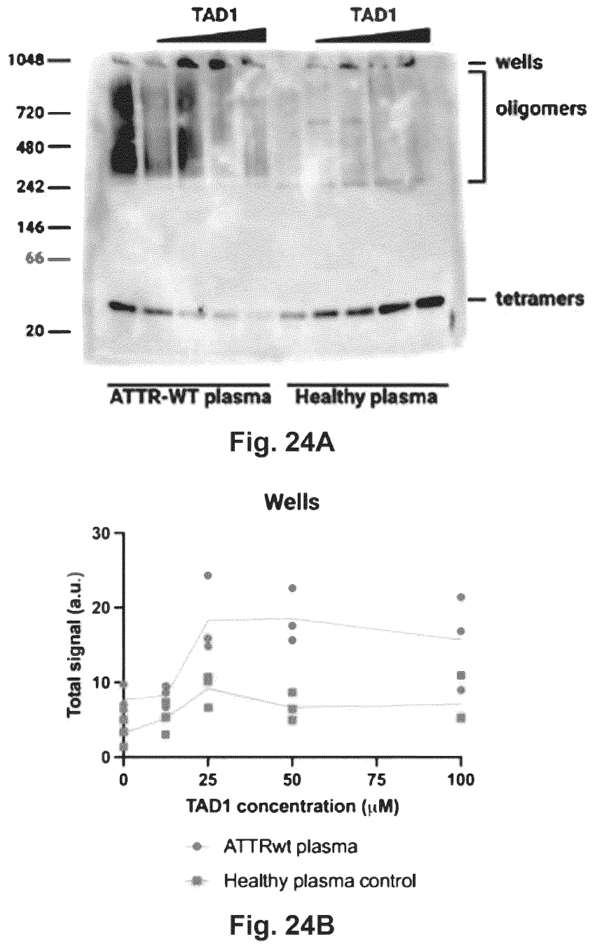

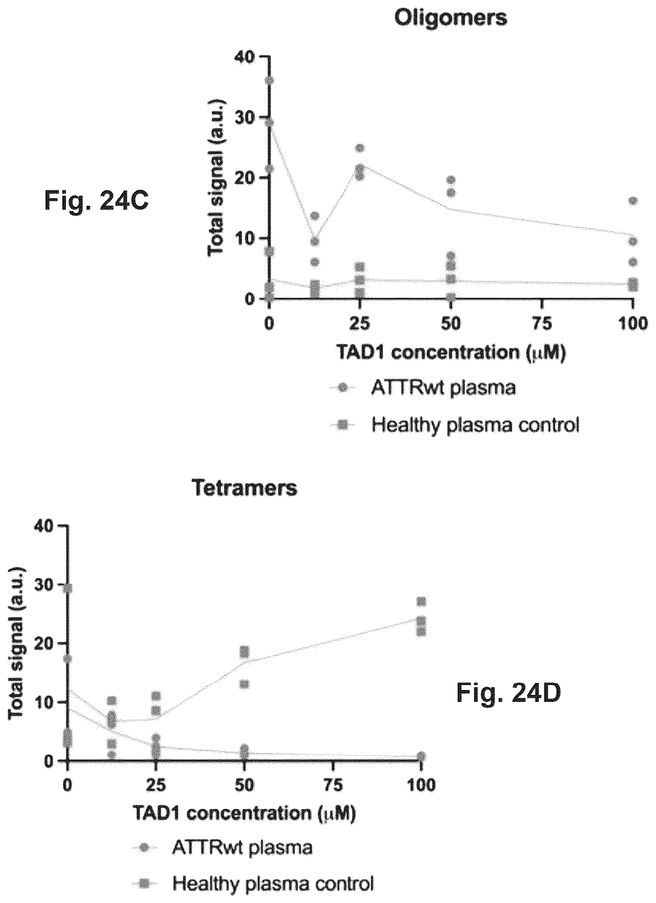

To characterize the binding of TAD1 to ATTR species in plasma, two complementary assays were performed. First, ATTR patient and healthy plasma were filtered using a 0.22 μM pore filter and assessed with dot blotting to measure TAD1 binding to species in the filtrate and the void (FIG. 22C). TAD1 binds aggregated high molecular weight TTR in plasma that cannot pass through the filter (FIG. 22D). In a second assay a non-denaturing protein-protein band shift experiment was conducted, which enables visualization of changes in electrophoretic behavior of a protein (soluble or aggregated) upon binding with a second protein (FIG. 24A). Briefly, ATTRwt patient plasma or negative control plasma were incubated with increasing concentrations of TAD1, run on a non-denaturing gel and probed with an anti-TTR antibody. The first interesting observation from this experiment was the presence of oligomeric ATTR species in the ATTRwt plasma, absent in the negative control (FIGS. 24A-24C). Upon binding to TAD1, these oligomers as well as tetrameric soluble transthyretin disappeared in a concentration-dependent manner. In contrast, high molecular weight species that do not run through the gel accumulated (FIG. 24A-248). It was also observed that there is an increase in tetrameric soluble transthyretin with increasing concentrations of TAD1 in healthy plasma (FIG. 24A, FIG. 24D) Together, these results indicate that TAD1 recognizes aggregated ATTR species that have a high molecular weight in a conformation-dependent manner.

### Example 19: TAD1 Binds ATTR Via a Unique Mechanism

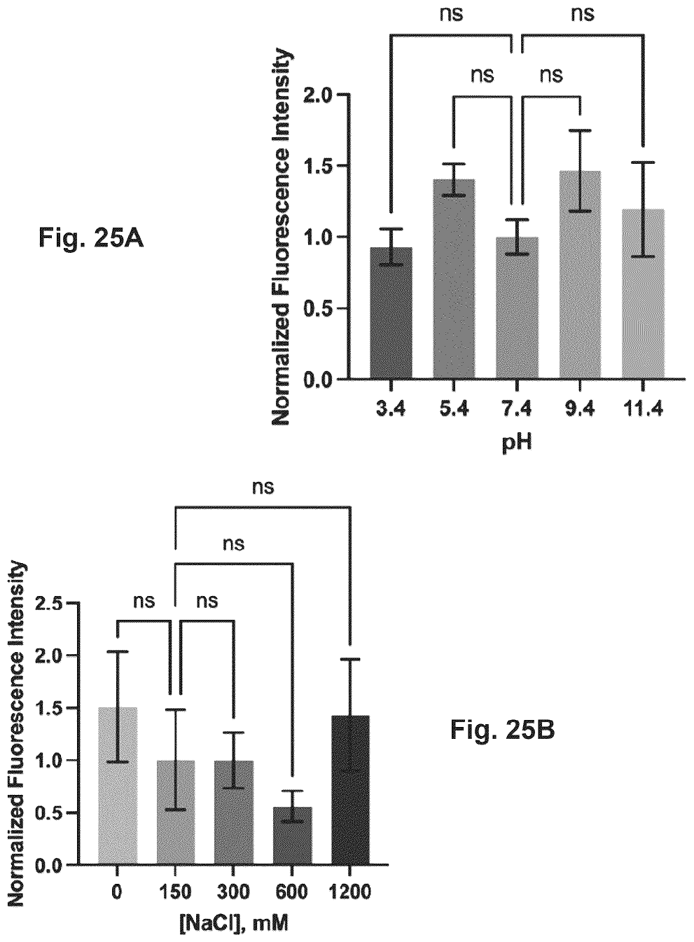

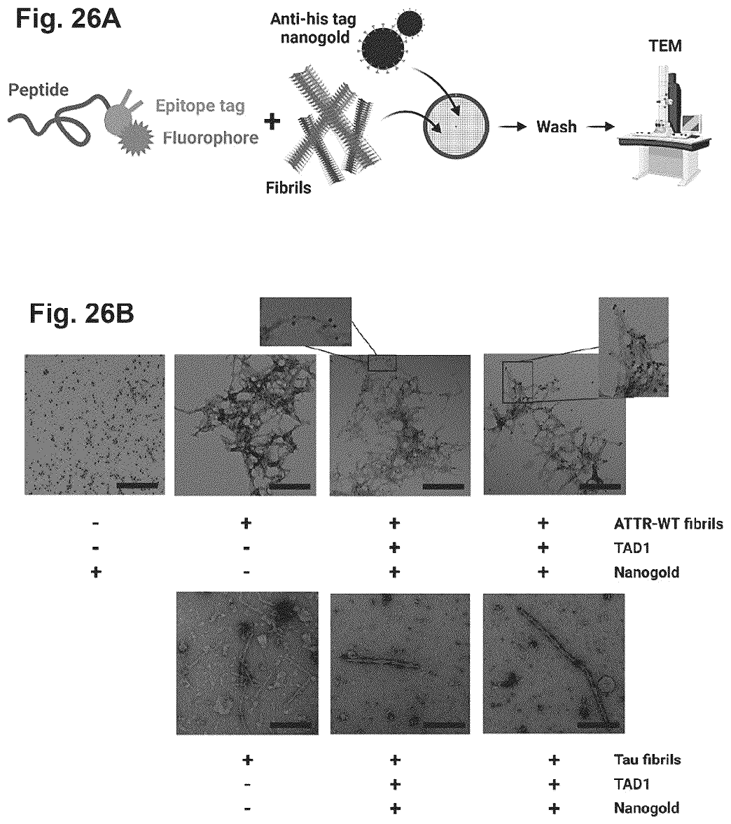

Finally, how and where TAD1 binds ATTR fibrils was evaluated. First, whether the interaction of TAD1 to ATTR species is electrostatic was tested because 18 out of 43 residues of TAD1 are charged amino acids under these experimental conditions. To test this hypothesis, TAD1 binding was tested under multiple pHs (FIG. 25A) and concentrations of salt (FIG. 25B). It was found that neither of these changes affected binding of TAD1 to purified ATTRwt fibrils significantly, suggesting that the interaction may be hydrophobic. Further experiments then tested where TAD1 interaction to purified ATTRwt fibrils take place. The poly-histidine tag present in TAD1 was exploited to coat this peptide with nickel nitrilotriacetic acid nanogold beads upon binding to purified ATTR fibrils (FIG. 26A). It was observed that the nanogold particles decorate ATTR fibrils primarily at the tips at low concentrations as well as on the surface of fibrils at higher concentrations (FIG. 26B). Tau fibrils were used as a negative control (FIG. 26C). The sensitive binding of nanogold particles to the tip of the fibrils is consistent with our structure-based peptide design pipeline (FIGS. 16A-16C). These experiments suggest that TAD1 binds ATTR species probably via hydrophobic interactions with the tip and the surface of fibrils.

### Example 20: Discussion of Examples 1 to 19

ATTR amyloidosis is a fatal disease that is likely underdiagnosed due to its complex diagnostic process. In recent years, there has been an outstanding advancement in the development of therapeutic options for ATTR amyloidosis that are effective at halting disease progression when administered at early stages. Thus, improving the diagnostic process is essential for enabling early treatment and therefore lessening patient burden. Here, the structures of aggregation-driving segments of ATTR were used to design a peptide for the detection of cardiac ATTR fibrils and aggregates in cardiac tissues and plasma (FIGS. 16A-16C). This peptide robustly detects ATTR fibrils purified from heart and within cardiac lysates with high sensitivity, specificity, and precision (FIGS. 18A, 18B, 19A, 19B, 19C and 19D). The data presented above also reveals a novel biomarker in plasma consisting of high molecular weight ATTR species (FIGS. 21A, 21B, 22A, 228, 22C, 22D, 24A, and 24B).

This structure-based peptide detects a novel plasma biomarker in cardiac ATTR amyloidosis patients. TAD1 detects not only ATTR fibrils, but also unique ATTR species present in blood of ATTR amyloidosis patients that were not observed in age-matched healthy controls or patients with another form of systemic amyloidosis (FIGS. 21A and 21B). These species are high molecular weight aggregates that, as our results suggest, may accumulate in the blood prior to an individual's onset of symptoms (FIGS. 21A, 21B, 22A, 22B, 22C, and 22D). Combined with previous results, these Examples indicate that there may be multiple types of non-native transthyretin species in the blood of ATTR amyloidosis patients, including monomeric misfolded states and high molecular weight aggregates (FIGS. 21A, 21B, 22A, 22B, 22C, and 22D). Perhaps the decrease of soluble transthyretin observed in patient serum is associated with the formation of these insoluble species that can be detected in plasma. Our study establishes a novel plasma biomarker for cardiac ATTR amyloidosis and opens questions about the basic biology of ATTR amyloid formation in the blood.

Current blood biomarkers for ATTR amyloidosis have limitations that the disclosed polypeptide probes address. Biomarkers such as natriuretic peptides and cardiac troponins are not specific for diagnosing cardiac ATTR amyloidosis, but general cardiac injury. The reliability and specificity of other biomarkers such as transthyretin and retinol binding protein 4 is still unknown. Groups have used segments of TTR to design novel peptides and antibodies that bind non-native TTR (NNTTR) in plasma of neuropathic patients but have limited efficacy in patients with purely cardiac phenotype or mixed phenotype. Another main challenge in using blood biomarkers for ATTR amyloidosis is distinguishing ATTR amyloidosis from other forms of systemic amyloidosis, such as immunoglobulin light chain amyloidosis (AL amyloidosis). The disclosed polypeptide probes robustly detect ATTR species in plasma of cardiac ATTR amyloidosis patients regardless of genotype or phenotype and distinguish between ATTR amyloidosis and AL amyloidosis (FIG. 21B). This strategy relies on the structures of ATTR fibrils to increase specificity, similar to what other groups have performed for the development of binders of other amyloid fibrils. Structure-based diagnostics are a potential avenue to simplify the diagnostic process for cardiac ATTR amyloidosis and detect the structural shift between native TTR and its matured amyloidogenic conformation.

TAD1 can potentially be used for the detection of cardiac ATTR amyloidosis prior to an individual's onset of symptoms (FIGS. 21A, 21B). The detection of ATTR species in the blood of ATTRv carriers without clinical manifestations of disease suggests that transthyretin aggregation starts in the blood (FIGS. 21A, 21B, 22A, 22B, 22C, and 22D). It has also shown that the misfolding of TTR starts in the blood, as shown in neuropathic patients. Early detection of TTR misfolding and ATTR aggregation in the blood may allow close monitoring of disease progression, thereby informing about the need for early treatment. The correlation between TTR misfolding and ATTR aggregation in the blood and the development of clinical phenotype will need to be established using conventional diagnostic methods to justify treatment.

Additionally, TAD1 can potentially be used to monitor treatment response or to optimize effective doses of a therapeutic. TAD1 fluorescence intensity decreases in the treatment group compared to the non-treatment group (FIGS. 21A and 21B). This result suggests that therapeutics designed to kinetically stabilize transthyretin lessen high molecular weight aggregates in the blood. Similar results are observed when examining the small group of patients on gene silencers, indicating that these treatments may also result in the reduction of ATTR aggregates in the blood. Studies detecting TTR misfolding in neuropathic patients after treatment with stabilizers, gene silencers, and liver transplantation also show a decrease in the presence of these misfolded species. These examples therefore describe a new biomarker of cardiac ATTR amyloidosis that can be exploited to monitor treatment response or to optimize treatment plans.

In summary, the structures of ATTR fibrils are used herein to design a novel peptide for the detection of ATTR fibrils and aggregates within cardiac ATTR amyloidosis patients. This peptide has revealed a novel plasma biomarker consisting of high molecular weight aggregated transthyretin. This peptide can detect these aggregates in the blood of ATTRv carriers prior to showing symptoms, indicating that can potentially be used for early detection. An observed reduction of signal in patients that received treatment further indicates that this biomarker could be used to monitor treatment response. Finally, this peptide can be used for the study of the biology and pathogenesis of ATTR amyloidosis, which may result in the identification of new targets for therapeutic development.

### Example 21: Identifying the Role of Transthyretin Aggregation in Preeclampsia

Preeclampsia (PE) is a pregnancy-specific vascular syndrome that emerges after 20 weeks of gestation and is a major cause of maternal, fetal and neonatal morbidity and mortality. Despite affecting approximately 2-8% of pregnancies worldwide, PE still lacks reliable, early diagnosis or therapy. Transthyretin (TTR) aggregates have been detected in the placenta of preeclamptic patients. These aggregates could represent a causal pathway for preeclampsia. In this example, experiments were performed to identify TTR in the placenta and correlate with maternal plasma levels for feasibility of biomarker use. It was hypothesized that preeclamptic plasma would demonstrate higher levels of TTR aggregates.

Levels of transthyretin (TTR) aggregates were measured in both placental lysates and maternal blood plasma by detecting fluorescent intensity through immunodot blotting and quantification with ImageJ. These measurements were compared among pregnancies without complication, complicated by chronic hypertension, chronic hypertension with superimposed PE with severe features, and with PE with severe features only. TTR aggregates were extracted and isolated from placental tissue and analyzed with Western blots and imaged under transmission electron microscopy.

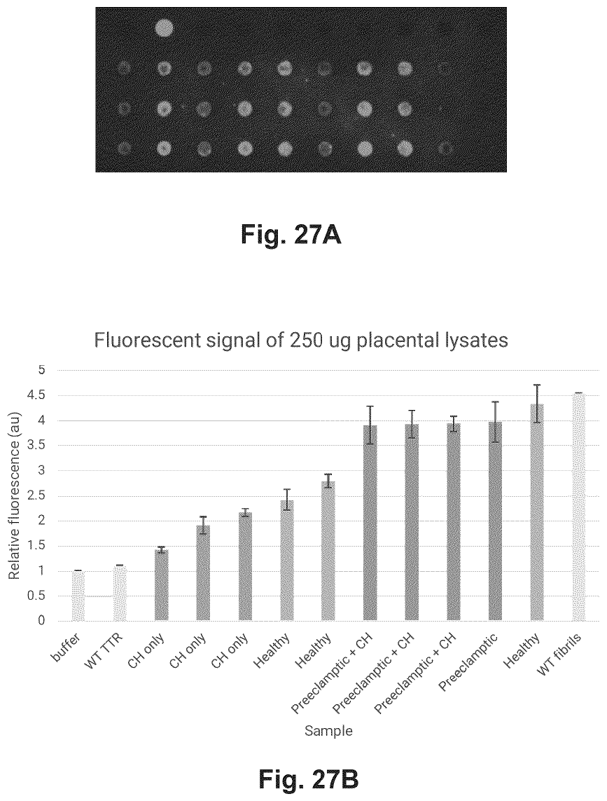

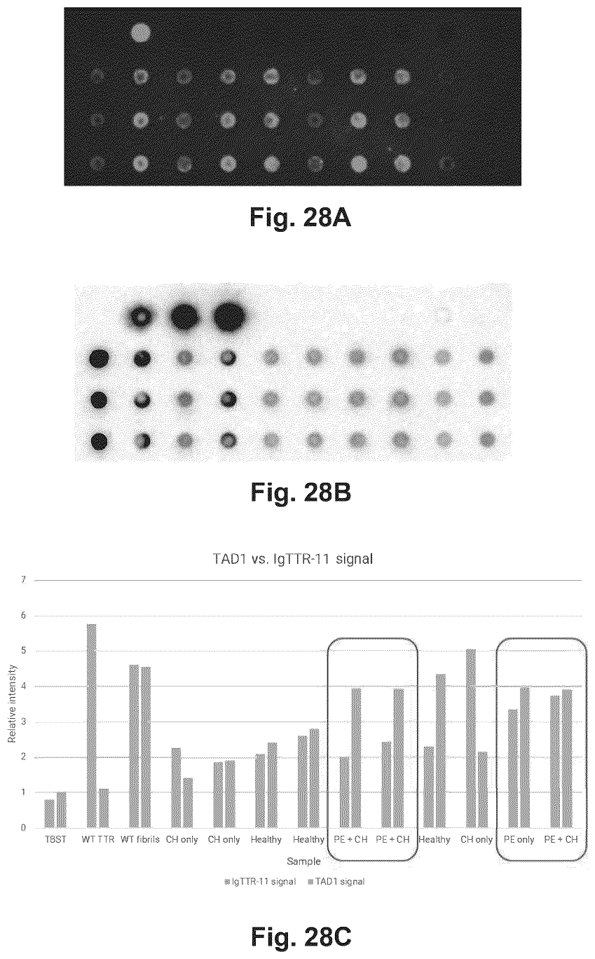

FIG. 27A shows a representative dot blot prepared from different placental lysates probed with TAD1, as described above. As quantified in FIG. 27B, placental lysates isolated from preeclamptic patients showed a significantly higher levels of TAD1 positive (e.g., aggregated) transthyretin species. These samples were then further analyzed to measure levels of aggregated TTR (e.g., TAD1 positive species) vs total TTR (as detected by a general anti-TTR antibody) and it was found that samples isolated from preeclamptic patients (PE) showed significantly higher levels of TAD1 (aggregated TTR) relative to total TTR (FIGS. 28A-28C). Specifically, the fluorescent signal of aggregated TTR species in preeclamptic placentas was 1.57 times higher than in those from pregnancies without preeclampsia.

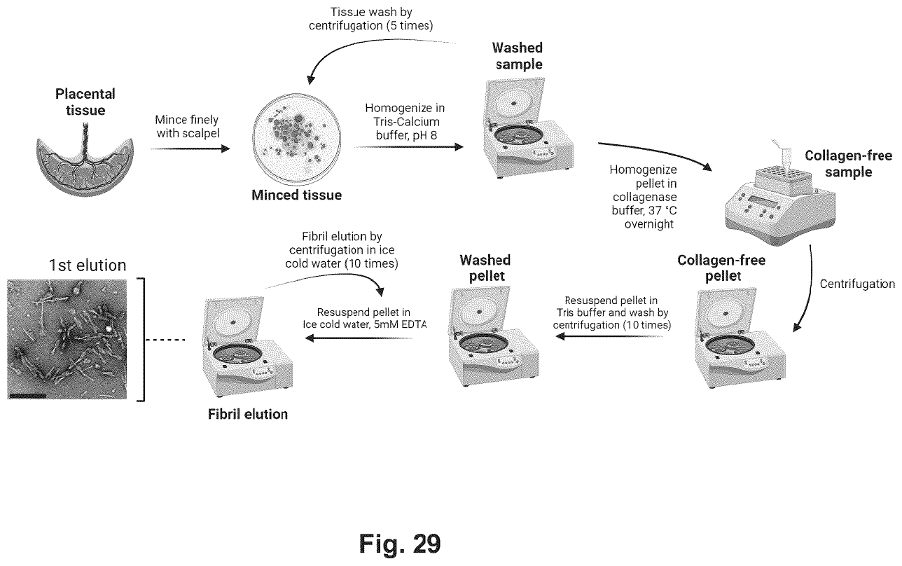

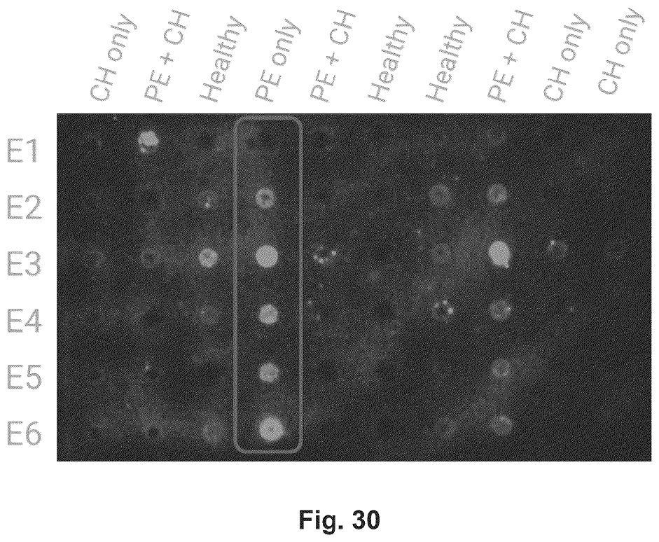

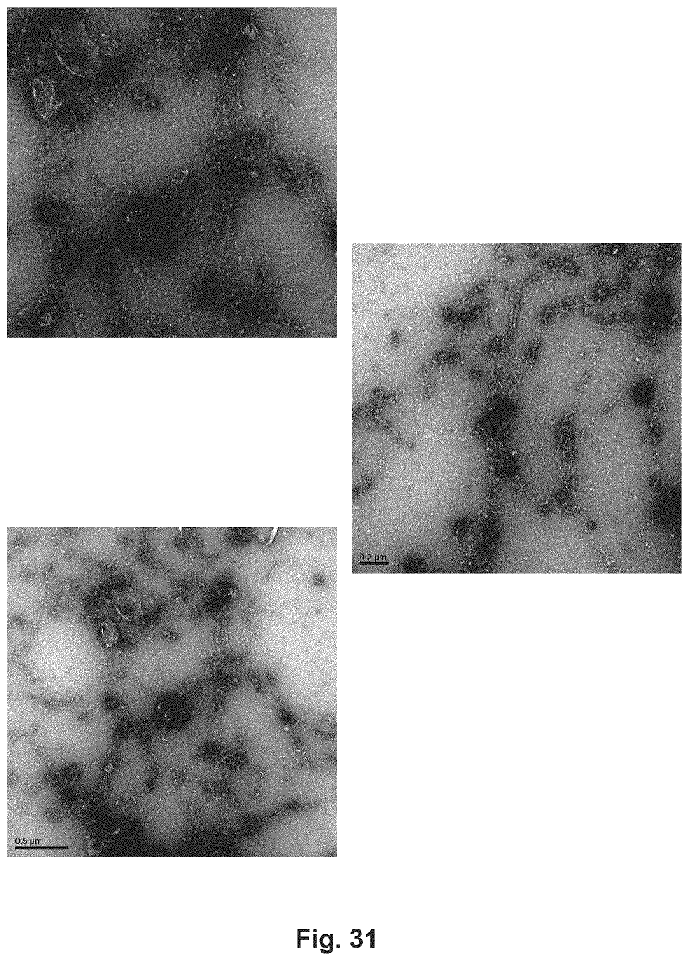

Fibrils were extracted from patient samples according to a workflow depicted in FIG. 29. Briefly, placental tissue was minced finely with a scalpel and homogenization and washed by centrifugation with a Tris-Calcium buffer (pH 8). The resulting pellet was homogenized in collagenase buffer at 37° C. overnight to generate a collagen-free sample, which was centrifuged to generate a collagen-free pellet. This pellet was resuspended in Tris buffer and washed with centrifugation to generate a washed pellet. This pellet was resuspended in ice cold water with 5 mM EDTA and centrifuged, repeatedly (10 times) to elute fibrils. FIG. 30 shows a representative dot plot of isolated fibrils from various samples probed with TAD1 and FIG. 31 shows illustrative brightfield images of isolated fibrils from preeclamptic patients.

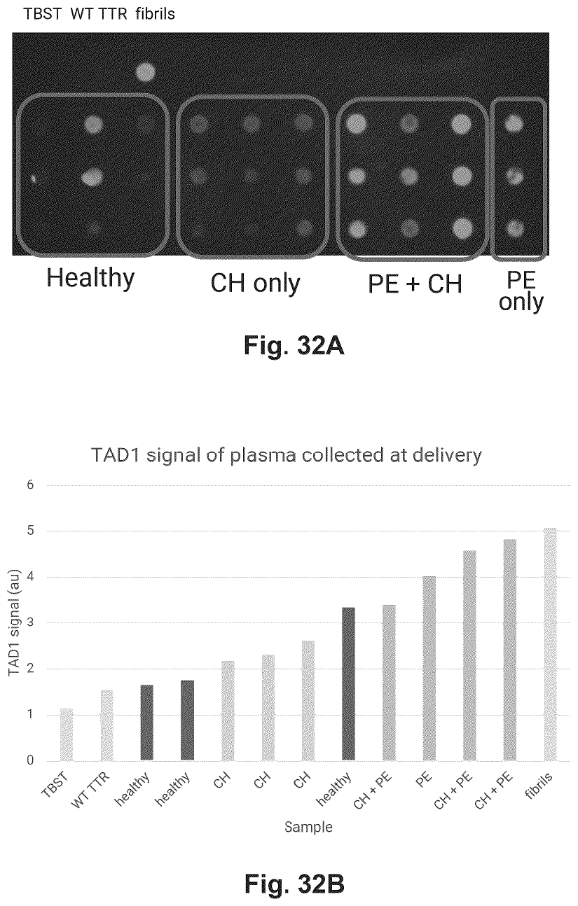

In a further experiment, TAD1 was used to detect TTR fibrils and aggregates in patient plasma collected at delivery. It was surprisingly found that aggregated TTR was elevated in preeclamptic patients (FIGS. 32A-32B). Specifically, the fluorescent signal in maternal plasma at delivery was, on average, 1.82 times higher in preeclamptic patients compared to non-preeclamptic plasma.

In summary, the foregoing data shows elevated levels of non-native TTR species in the placentas and blood plasma of preeclamptic patients. The association of higher levels of aggregated transthyretin observed in preeclamptic plasma suggests application as a novel biomarker.

The Table below lists Sequences provided in this Application.

| | | SEQ |
|---|---|---|
| | | ID |
| Name | Sequence | NO: |
| First or second peptide | HVAHPFVEFTE | 1 |
| First or second peptide | SYVTNPTSYAVT | 2 |
| Linker | GGGSTE | 3 |
| Linker | EAAAK | 4 |
| Linker | PAPAP | 5 |
| Linker | GGGGGG | 6 |
| Epitope tag | DYKDDDDK | 7 |
| Epitope tag | YPYDVPDYA | 8 |
| Peptide + peptide + arg | RRRRHVAHPFVEFTEGGG | 9 |
| tags | STERRRRSYVTNPTSYAVT | |
| Peptide + peptide + arg | YPYDVPDYARRRRHVAHP | 10 |
| tags + epitope | FVEFTEGGGSTERRRRSY | |
| | VTNPTSYAVT | |
| Peptide + peptide + arg | DYKDDDDKRRRRHVAHPF | 11 |
| tags + eptitope | VEFTEGGGSTERRRRSYV | |
| | TNPTSYAVT | |
| Peptide + peptide + arg | HHHHHHRRRRHVAHPFVE | 12 |
| tags + epitope | FTEGGGSTERRRRSYVTN | |
| | PTSYAVT | |
| Tab3-01 | RRRRHVAHPFVEFTE | 13 |
| Full probe | TAMRA -YPYDVPDYA- | 14 |
| | RRRRHVAHPFVEFTEGGG | |
| | STERRRRSYVTNPTSYAVT | |
| Full probe | TAMRA-DYKDDDDK- | 15 |
| | RRRRHVAHPFVEFTEGGG | |
| | STERRRRSYVTNPTSYAVT | |
| Full probe | TAMRA-HHHHHH- | 16 |
| | RRRRHVAHPFVEFTEGGG | |
| | STERRRRSYVTNPTSYAVT | |
| Full probe | FITC -Ahx - | 17 |
| | YPYDVPDYA- | |
| | RRRRHVAHPFVEFTEGGG | |
| | STERRRRSYVTNPTSYAVT | |
| Full probe | FITC-Ahx-DYKDDDDK- | 18 |
| | RRRRHVAHPFVEFTEGGG | |
| | STERRRRSYVTNPTSYAVT | |
| Full probe | FITC-Ahx-HHHHHH- | 19 |
| | RRRRHVAHPFVEFTEGGG | |
| | STERRRRSYVTNPTSYAVT | |
| Full probe | FITC-Ahx- | 20 |
| | RRRRHVAHPFVEFTEGGG | |
| | STERRRRSYVTNPTSYAVT | |
| Tab3-02 | RRRRSYVTNPTSYAVT | 21 |
| TTR fibril peptide | VAVHVF | 22 |
| (Strand B) | | |
| TTR fibril peptide | IYKVEI | 23 |
| (Strand E) | | |
| TTR fibril peptide | KALGIS | 24 |
| (Loop connecting | | |
| Strands E and F) | | |
| TTR fibril peptide | AEVVFT | 25 |
| (Strand F) | | |
| TTR fibril peptide | YTIAAL | 26 |
| (Strand G) | | |
| TTR fibril peptide | TIALLS | 27 |
| (Strand G) | | |
| TTR fibril peptide | TAVVTN | 28 |
| (Strand H) | | |
| Plurality of Arginine | RRRR | 29 |
| residues | | |
| Plurality of Histidine | HHHHHH | 30 |
| residues | | |
| Plurality of Histidine | HHHHHH-RRRR | 31 |
| and Arginine | | |
| Residues | | |
| Tab3-03 | RRRRHVAHPFVEFTERR | 32 |
| | RRSYVTNPTSYAVT | |
| Tab3-04 | RRRRSYVTNPTSYAVTR | 33 |
| | RRRHVAHPFVEFTE | |
| Tab3-05 | RRRRHVAHPFV*EFTER | 34 |
| | RRRSYVTNPTSY*AVT | |
| Tab3-06 | RRRRSYVTNPTSY*AVT | 35 |
| | RRRRHVAHPFV*EFTE | |
| Tab3-07 | RRRRHVAHPFVEFTEGS | 36 |
| | RRRRSYVTNPTSYAVT | |
| Tab3-08 | RRRRHVAHPFVEFTEGG | 37 |
| | SRRRRSYVTNPTSYAVT | |
| Tab3-09 | RRRRHVAHPFVEFTEGG | 38 |
| | GSRRRRSYVTNPTSYAV | |
| | T | |
| Tab3-10 | RRRRHVAHPFVEFTEGG | 39 |
| | GSTRRRRSYVTNPTSYA | |
| | VT | |
| Tab3-11 | RRRRHVAHPFVEFTEGG | 40 |
| | GSARRRRSYVTNPTSYA | |
| | VT | |
| Tab3-13 | RRRRHVAHPFVEFTEGG | 41 |
| | GSAERRRRSYVTNPTSY | |
| | AVT | |

*N-methyl modification on the preceding residue

1. A method of determining whether a subject is at risk of preeclampsia, the method comprising: (a) detecting a transthyretin oligomer, fibril or molecule in a sample obtained from the subject, and (b) identifying the subject as at risk for preeclampsia if the transthyretin oligomer, fibril or molecule is detected in the sample.

2. The method of claim 1, wherein the subject is determined to be at risk of preeclampsia if a level of transthyretin oligomer, fibril or molecule detected in the sample exceeds a threshold.

3. A method of diagnosing a subject with preeclampsia, the method comprising: (a) detecting a transthyretin oligomer, fibril or molecule in a sample obtained from the subject and (b) diagnosing the subject with preeclampsia if the transthyretin oligomer, fibril or molecule is detected in the sample.

4. The method of claim 3, wherein the subject is diagnosed with preeclampsia or if a level of transthyretin oligomer, fibril or molecule detected in the sample exceeds a threshold.

5. A method of monitoring an effectiveness of a therapeutic administered to a subject to treat a preeclampsia, the method comprising (a) detecting an oligomer, aggregate or fibril of transthyretin in a first sample obtained from the subject, (b) administering the therapeutic to the subject, and (c) detecting an oligomer, aggregate or fibril of transthyretin in a second sample obtained from the sample after the therapeutic is administered, wherein the therapeutic is determined to be effective if fewer oligomers, aggregates and/or fibrils of transthyretin are detected in the second sample compared to the first sample.

6. The method of claim 5, further comprising monitoring more than one dose of the therapeutic to identify an effective amount of the therapeutic, wherein the effective amount of the therapeutic results in a larger reduction in the detection of oligomers, aggregates, and/or fibrils of transthyretin in the second sample compared to the first sample.

7. The method of claim 5 or 6, wherein the subject has been determined to be at risk for preeclampsia according to the method of claim 1 and/or diagnosed with preeclampsia according to the method of claim 3.

8. A method of treating a subject for preeclampsia, the method comprising administering an effective amount of a therapeutic to the subject, wherein (a) the subject has been determined to be at risk for preeclampsia according to claim 1, (b) the subject has been diagnosed with preeclampsia according to claim 3, and/or (c) the effective amount of the therapeutic is determined according to the method of claim 5.

9. The method of any one of claims 5 to 8, wherein the therapeutic comprises an inhibitor of transthyretin expression and/or aggregation.

10. The method of claim 9, wherein the therapeutic comprises a small molecule, a gene silencer or an antibody.

11. The method of claim 10, wherein the therapeutic comprises tafamidis.

12. The method of any one of claims 1 to 11, wherein detecting the transthyretin oligomer, fibril or molecule comprises (a) contacting a sample first with a polypeptide probe selective for aggregated transthyretin, (b) allowing the polypeptide probe to bind any oligomers, aggregates or fibrils of transthyretin in the sample, and (c) detecting a complex comprising the polypeptide probe and an oligomer, aggregate or fibril of transthyretin, wherein the presence of the complex correlates to the presence of an oligomer, aggregate or fibril of transthyretin in the sample.

13. The method of claim 12, wherein the polypeptide probe selective for aggregated transthyretin comprises first peptide comprising the sequence HVAHPFVEFTE (SEQ ID NO: 1) and a second peptide comprising the sequence SYVTNPTSYAVT (SEQ ID NO: 2), wherein the first and second peptide are covalently linked via a linker peptide and wherein the polypeptide probe further comprises a detectable label.

14. The method of claim 13, wherein the linker peptide comprises the sequence GGGSTE (SEQ ID NO: 3), EAAAK (SEQ ID NO: 4), PAPAP (SEQ ID NO: 5) or GGGGGG (SEQ ID NO: 6).

15. The method of claim 13 or 14, wherein the polypeptide probe further comprises an epitope tag that facilitates solubility, manipulation and/or purification of the polypeptide.

16. The method of claim 15, wherein the epitope tag comprises a peptide that increases the affinity of the polypeptide to an affinity column.

17. The method of claim 15 or 16, wherein the epitope tag comprises a plurality of histidine or lysine residues.

18. The method of any one of claims 15 to 17, wherein the epitope tag comprises a peptide consisting of the sequence DYKDDDDK (SEQ ID NO: 7) or YPYDVPDYA (SEQ ID NO: 8).

19. The method of any one of claims 15 to 18, wherein the epitope tag increases the solubility of the polypeptide.

20. The method of claim 19, wherein the epitope tag comprises a plurality of arginine residues.

21. The method of any one of claims 13 to 20, wherein the detectable label is covalently linked to the N-terminus of the first peptide or to the C-terminus of the second peptide.

22. The method of any one of claims 13 to 21, wherein the detectable label is covalently linked to polypeptide probe via a linker.

23. The method of claim 22, wherein the linker comprises aminohexanoic acid (Ahx).

24. The method of any one of claims 13 to 23, wherein the detectable label comprises tetramethylrhodamine (TAMRA), Fluorescein isothiocyanate (FITC), aminohexanoic acid (Ahx) or any combination thereof.

25. The method of any one of claims 13 to 24, wherein the polypeptide probe comprises the amino acid sequence RRRRHVAHPFVEFTEGGGSTERRRRSYVTNPTSYAVT (SEQ ID NO: 9)

26. The method of any one of claims 13 to 25, wherein the polypeptide probe comprises the amino acid sequence YPYDVPDYARRRRHVAHPFVEFTEGGGSTERRRRSYVTNPTSYAVT (SEQ ID NO: 10), DYKDDDDK-RRRRHVAHPFVEFTEGGGSTERRRRSYVTNPTSYAVT (SEQ ID NO: 11), or HHHHHHRRRRHVAHPFVEFTEGGGSTERRRRSYVTNPTSYAVT (SEQ ID NO: 12).

27. The method of any one of claims 13 to 26, wherein the polypeptide probe is selected from the group consisting of: TAMRA-YPYDVPDYA-RRRRHVAHPFVEFTEGGGSTERRRRSYVTNPTSYAVT (SEQ ID NO: 14), TAMRA-DYKDDDDK-RRRRHVAHPFVEFTEGGGSTERRRRSYVTNPTSYAVT (SEQ ID NO: 15), TAMRA-HHHHHH-RRRRHVAHPFVEFTEGGGSTERRRRSYVTNPTSYAVT (SEQ ID NO: 16), FITC-Ahx-YPYDVPDYA-RRRRHVAH PFVEFTEGGGSTERRRRSYVTNPTSYAVT (SEQ ID NO: 17), FITC-Ahx-DYKDDDDK-RRRRHVAHPFVEFTEGGGSTERRRRSYVTNPTSYAVT (SEQ ID NO: 18), FITC-Ahx-HHHHHH-RRRRHVAHPFVEFTEGGGSTERRRRSYVTNPTSYAVT (SEQ ID NO: 19), FITC-Ahx-RRRRHVAHPFVEFTEGGGSTERRRRSYVTNPTSYAVT (SEQ ID NO: 20) wherein TAMRA is tetramethylrhodamine, FITC is Fluorescein isothiocyanate (FITC) and Ahx is an aminohexanoic acid linker.

28. The method of any one of claims 13 to 27, wherein the polypeptide probe is FITC-Ahx-HHHHHH-RRRRHVAHPFVEFTEGGGSTERRRRSYVTNPTSYAVT (SEQ ID NO: 19), FITC-Ahx-YPYDVPDYA-RRRRHVAHPFVEFTEGGGSTERRRRSYVTNPTSYAVT (SEQ ID NO: 17), or FITC-Ahx-DYKDDDDK-RRRRHVAHPFVEFTEGGGSTERRRRSYVTNPTSYAVT (SEQ ID NO: 18), wherein FITC is Fluorescein isothiocyanate (FITC) and Ahx is an aminohexanoic acid linker.

29. The method of any one of claims 13 to 28, wherein the polypeptide probe is FITC-Ahx-H HHHHH-RRRRHVAH PFVEFTEGGGSTERRRRSYVTNPTSYAVT (SEQ ID NO: 19), wherein FITC is Fluorescein isothiocyanate (FITC) and Ahx is an aminohexanoic acid linker.


SEQUENCE LISTING (41 sequences)

SEQ ID NO: 1 - protein, 11 aa, synthetic construct
HVAHPFVEFTE

SEQ ID NO: 2 - protein, 12 aa, synthetic construct
SYVTNPTSYAVT

SEQ ID NO: 3 - protein, 6 aa, synthetic construct
GGGSTE

SEQ ID NO: 4 - protein, 5 aa, synthetic construct
EAAAK

SEQ ID NO: 5 - protein, 5 aa, synthetic construct
PAPAP

SEQ ID NO: 6 - protein, 6 aa, synthetic construct
GGGGGG

SEQ ID NO: 7 - protein, 8 aa, synthetic construct
DYKDDDDK

SEQ ID NO: 8 - protein, 9 aa, synthetic construct
YPYDVPDYA

SEQ ID NO: 9 - protein, 37 aa, synthetic construct
RRRRHVAHPFVEFTEGGGSTERRRRSYVTNPTSYAVT

SEQ ID NO: 10 - protein, 46 aa, synthetic construct
YPYDVPDYARRRRHVAHPFVEFTEGGGSTERRRRSYVTNPTSYAVT

SEQ ID NO: 11 - protein, 45 aa, synthetic construct
DYKDDDDKRRRRHVAHPFVEFTEGGGSTERRRRSYVTNPTSYAVT

SEQ ID NO: 12 - protein, 43 aa, synthetic construct
HHHHHHRRRRHVAHPFVEFTEGGGSTERRRRSYVTNPTSYAVT

SEQ ID NO: 13 - protein, 15 aa, synthetic construct
RRRRHVAHPFVEFTE

SEQ ID NO: 14 - protein, 46 aa, synthetic construct; identical to SEQ ID NO: 10
  UNSURE 1: note = TAMRA (tetramethylrhodamine) label

SEQ ID NO: 15 - protein, 45 aa, synthetic construct; identical to SEQ ID NO: 11
  UNSURE 1: note = TAMRA (tetramethylrhodamine) label

SEQ ID NO: 16 - protein, 43 aa, synthetic construct; identical to SEQ ID NO: 12
  UNSURE 1: note = tetramethylrhodamine (TAMRA) label

SEQ ID NO: 17 - protein, 46 aa, synthetic construct; identical to SEQ ID NO: 10
  UNSURE 1: note = Fluorescein isothiocyanate (FITC) -Ahx (aminohexanoic acid) label

SEQ ID NO: 18 - protein, 45 aa, synthetic construct; identical to SEQ ID NO: 11
  UNSURE 1: note = Fluorescein isothiocyanate (FITC) -Ahx (aminohexanoic acid) label

SEQ ID NO: 19 - protein, 43 aa, synthetic construct; identical to SEQ ID NO: 12
  UNSURE 1: note = Fluorescein isothiocyanate (FITC) -Ahx (aminohexanoic acid) label

SEQ ID NO: 20 - protein, 37 aa, synthetic construct; identical to SEQ ID NO: 9
  UNSURE 1: note = Fluorescein isothiocyanate (FITC)- aminohexanoic acid (Ahx) label

SEQ ID NO: 21 - protein, 16 aa, synthetic construct
RRRRSYVTNPTSYAVT

SEQ ID NO: 22 - protein, 6 aa, synthetic construct
VAVHVF

SEQ ID NO: 23 - protein, 6 aa, synthetic construct
IYKVEI

SEQ ID NO: 24 - protein, 6 aa, synthetic construct
KALGIS

SEQ ID NO: 25 - protein, 6 aa, synthetic construct
AEVVFT

SEQ ID NO: 26 - protein, 6 aa, synthetic construct
YTIAAL

SEQ ID NO: 27 - protein, 6 aa, synthetic construct
TIALLS

SEQ ID NO: 28 - protein, 6 aa, synthetic construct
TAVVTN

SEQ ID NO: 29 - protein, 4 aa, synthetic construct
RRRR

SEQ ID NO: 30 - protein, 6 aa, synthetic construct
HHHHHH

SEQ ID NO: 31 - protein, 10 aa, synthetic construct
HHHHHHRRRR

SEQ ID NO: 32 - protein, 31 aa, synthetic construct
RRRRHVAHPFVEFTERRRRSYVTNPTSYAVT

SEQ ID NO: 33 - protein, 31 aa, synthetic construct
RRRRSYVTNPTSYAVTRRRRHVAHPFVEFTE

SEQ ID NO: 34 - protein, 31 aa, synthetic construct; identical to SEQ ID NO: 32
  MOD_RES 11: note = N-methylated
  MOD_RES 28: note = N-methylated

SEQ ID NO: 35 - protein, 31 aa, synthetic construct; identical to SEQ ID NO: 33
  MOD_RES 13: note = N-methylated
  MOD_RES 27: note = N-methylated

SEQ ID NO: 36 - protein, 33 aa, synthetic construct
RRRRHVAHPFVEFTEGSRRRRSYVTNPTSYAVT

SEQ ID NO: 37 - protein, 34 aa, synthetic construct
RRRRHVAHPFVEFTEGGSRRRRSYVTNPTSYAVT

SEQ ID NO: 38 - protein, 35 aa, synthetic construct
RRRRHVAHPFVEFTEGGGSRRRRSYVTNPTSYAVT

SEQ ID NO: 39 - protein, 36 aa, synthetic construct
RRRRHVAHPFVEFTEGGGSTRRRRSYVTNPTSYAVT

SEQ ID NO: 40 - protein, 36 aa, synthetic construct
RRRRHVAHPFVEFTEGGGSARRRRSYVTNPTSYAVT

SEQ ID NO: 41 - protein, 37 aa, synthetic construct
RRRRHVAHPFVEFTEGGGSAERRRRSYVTNPTSYAVT